(19) United States
(12) Patent Application Publication
(10) Pub. No.: US 2022/0409647 A1
(43) Pub. Date: Dec. 29, 2022

(54) COMPOSITIONS, METHODS AND KITS FOR ALTERING ADIPOCYTES

(71) Applicant: NuSirt Sciences, Inc., Nashville, TN (US)

(72) Inventor: Michael B. Zemel, Knoxville, TN (US)

(21) Appl. No.: 17/740,970

(22) Filed: May 10, 2022

Related U.S. Application Data
(63) Continuation of application No. PCT/US2020/059879, filed on Nov. 10, 2020.
(60) Provisional application No. 62/933,895, filed on Nov. 11, 2019.

(51) Int. Cl. A61K 31/706; A61K 31/352

(52) U.S. Cl. CPC A61K 31/706; A61K 31/352

(57) ABSTRACT

Compositions, methods, and kits useful for treating obesity conditions are provided herein. Such compositions can contain synergizing amounts of nicotinamide riboside and/or nicotinamide mononucleotide and/or nicotinic acid metabolites in combination with a flavonoid and/or one or more flavonoid derivatives, with or without resveratrol.

### CROSS-REFERENCE

This application is a continuation of International Patent Application No. PCT/US2020/59879, filed Nov. 10, 2020, which claims the benefit of priority to U.S. Provisional Application No. 62/933,895, filed Nov. 11, 2019, each of which application is incorporated herein by reference in their entireties for all purposes.

### BACKGROUND OF THE INVENTION

Metabolic disorders, such as hyperlipidemia and obesity, and the related impact on health and mortality, present a significant burden to public health. For instance, obesity, clinically defined as a body mass index of over 30 kg/m², is estimated to affect 35.7% of the U.S. adult population. In the U.S., obesity is estimated to cause roughly 110,000-365,000 deaths per year. Obesity can result in hyperlipidemia, characterized by an excess of lipids, including cholesterol, cholesterol esters, phospholipids, and triglycerides, in the bloodstream, and can further result in diabetes, vascular disease, cancer, renal disease, infectious diseases, external causes, intentional self-harm, nervous system disorders, and chronic pulmonary disease (N Engl J Med 2011; 364:829-841). Metabolic Syndrome, in which subjects present with central obesity and at least two other metabolic disorders (such as high cholesterol, high blood pressure, or diabetes), is estimated to affect 25% of the U.S. population.

Certain metabolic disorders, such as obesity, can be treated with various medications, including nicotinic acid or its derivatives. Nicotinic acid is a form of vitamin B3 (niacin). When taken in high doses (1-3 g/day), nicotinic acid can reduce serum lipids and positively impact energy metabolism.

However, nicotinic acid can have a significant side-effect and hence can be generally poorly tolerated. One significant side-effect can be severe cutaneous vasodilation and flushing responses, and is consequently infrequently prescribed despite well documented safety and efficacy (Carlson La. Nicotinic acid: the broad-spectrum lipid drug. A 50th anniversary review. J Int Med 2005; 258:94-114). While side effects are somewhat attenuated in sustained (SR) and extended (ER) release preparations, the side effects persist sufficiently to limit drug use. Beneficial effects of nicotinic acid result, in part, from increasing intracellular NAD⁺ and thereby activating sirtuins. Nicotinamide mononucleotide (NMN) and nicotinamide riboside (NR) are two forms of vitamin B3 that have been demonstrated to increase intracellular NAD⁺ in preclinical studies. NR has been shown to exert this effect in humans as well, while data from NMN are not yet available. However, both human and animal data indicate that the increase in NAD⁺ resulting from administration of either compound is ˜40%, resulting in NAD⁺ levels well below the Km for Sirt1 activation. Accordingly, there exists a need to augment the efficacy of the NAD⁺ donors to achieve optimal Sirt1 activation.

### SUMMARY OF THE INVENTION

The subject application provides compositions, methods, and kits for stimulating browning in human fat cells (adipocytes), with resulting benefits including reductions in body weight and body fat; improved insulin sensitivity; and decreased serum lipids. The present invention provides means of treating obesity, hyperlipidemia, prediabetes and/or diabetes in a subject. The compositions, methods, and kits can include naringenin and/or other members of the flavonoids in combination with a sirtuin pathway activator such as NR, NMN, or other nicotinic acid metabolites.

The subject compositions can be administered orally or through other routes such as intravenous administration. Compositions for oral administration can include pills, tablets, capsules, and the like.

In an aspect, a composition is provided comprising an amount of a flavonoid or a derivative thereof and an amount of a sirtuin pathway activator wherein the amount of (a) and the amount of (b) in combination are effective in enhancing in a subject at least one physiological effect selected from the group consisting of mitochondrial biogenesis, adipocyte beiging, thermogenesis, fatty acid oxidation, weight loss, fat loss, and insulin sensitivity.

In some cases, the enhanced adipocyte beiging is evidenced by an increase in an expression or an activity of a gene implicated in adipocyte beiging. In some cases, the gene implicated in adipocyte beiging is selected from the group consisting of UCP1, PRDM16, PCG1α, GLUT4, Cidea, Elovl3, Ppary, Cox8b, Dio2, Ndufs1, and Tbx1. In some cases, the expression or the activity of the gene implicated in adipocyte beiging is increased at least two-fold.

In some cases, the thermogenesis is evidenced by an increase in an expression or an activity of a gene implicated in thermogenesis. In some cases, the gene implicated in thermogenesis is selected from the group consisting of UCP1, PCG1α, PRDM16, PCG1β, Ppary, COX-2, and Cidea. In some cases, the expression or the activity of the gene implicated in thermogenesis is increased at least two-fold.

In some cases, the enhanced fatty acid oxidation is evidenced by an increase in an expression or an activity of a gene implicated in fatty acid oxidation, weight loss, or fat loss. In some cases, the gene implicated in fatty acid oxidation, weight loss, or fat loss is selected from the group consisting of CPT1β, AdipoQ, ChREBP, ATGL, UCP2, CPT1α, and NPY. In some cases, the enhanced fatty acid oxidation is evidenced by an increase in oxygen consumption or a decrease in respiratory quotient, as measured by respiratory calorimetry. In some cases, the expression or the activity of the gene implicated in fatty acid oxidation, weight loss, or fat loss is increased at least two-fold.

In some cases, the enhanced insulin sensitivity is evidenced by an increase in an expression or an activity of a gene implicated in insulin sensitivity. In some cases, the gene implicated in insulin sensitivity is selected from the group consisting of GLUT4, AdipoQ, and ChREBP. In some cases, the enhanced insulin sensitivity is evidenced by a decrease in fasting insulin, a decrease in homeostatic assessment of insulin resistance (HOMA_{IR}), a decrease in 60-minute glucose or insulin in a glucose tolerance test (GTT), or a decrease in the glucose or insulin area under the curve in a GTT. In some cases, the expression or the activity of the gene implicated in insulin sensitivity is increased at least two-fold.

In some cases, the administration of (a) and (b) is in an amount that synergistically enhances adipocyte beiging. In some cases, the administration of (a) and (b) is in an amount that synergistically enhances thermogenesis. In some cases, the administration of (a) and (b) is in an amount that synergistically enhances fatty acid oxidation, weight loss, or fat loss. In some cases, the administration of (a) and (b) is in an amount that synergistically enhances insulin sensitivity.

In some cases, the flavonoid or derivative thereof comprises a flavanone compound. In some cases, the flavanone compound comprises a compound selected from the group consisting of blumeatin, butin, eriodictyol, hesperetin, hesperidin, homoeriodictyol, isosakuranetin, naringenin, naringin, pinocembrin, poncirin, sakuranetin, sakuranin, sterubin, and/or pinostrobin. In some cases, the sirtuin pathway activator comprises nicotinamide riboside, nicotinamide mononucleotide, nicotinic acid, or a nicotinic acid metabolite. In some cases, the nicotinic acid metabolite comprises nicotinyl CoA, nicotinuric acid, nicotinate mononucleotide, nicotinate adenine dinucleotide, or nicotinamide adenine dinucleotide. In some cases, the flavonoid comprises naringenin and the sirtuin pathway activator comprises nicotinamide riboside. In some cases, the molar ratio of (a) to (b) is at least 3:1.

In some cases, the subject comprises a human. In some cases, the amount of (b) is a sub-therapeutic amount when administered alone to the subject In some cases, the sirtuin pathway activator is substantially free of nicotinamide.

In another aspect, a method of enhancing in a subject one or more physiological effect selected from the group consisting mitochondrial biogenesis, adipocyte beiging, thermogenesis, fat oxidation, weight loss, fat loss, and insulin sensitivity is provided, comprising administering to the subject a composition of comprising a flavonoid or flavonoid derivative and a sirtuin pathway activator over a time period sufficient to yield an enhanced physiological effect.

In some cases, the enhancing of the adipocyte beiging is evidenced by an increase in an expression or an activity of a gene implicated in adipocyte beiging. In some cases, the gene implicated in adipocyte beiging is selected from the group consisting of UCP1, PRDM16, PCG1α, GLUT4, Cidea, Elovl3, Ppary, Cox8b, Dio2, and Tbx1. In some cases, the expression or the activity of the gene implicated in adipocyte beiging is increased at least two-fold.

In some cases, the thermogenesis is evidenced by an increase in an expression or an activity of a gene implicated in thermogenesis. In some cases, the gene implicated in thermogenesis is selected from the group consisting of UCP1, PCG1α, PRDM16, PCG1β, Ppary, COX-2, and Cidea. In some cases, the expression or the activity of the gene implicated in thermogenesis is increased at least two-fold.

In some cases, the enhanced fatty acid oxidation, weight loss, or fat loss is evidenced by an increase in an expression or an activity of a gene implicated in fatty acid oxidation, weight loss, or fat loss. In some cases, the gene implicated in fatty acid oxidation, weight loss, or fat loss is selected from the group consisting of CPT1β, AdipoQ, ChREBP, ATGL, UCP2, CPT1α, and NPY. In some cases, the enhanced fatty acid oxidation is evidenced by an increase in oxygen consumption or a decrease in respiratory quotient, as measured by respiratory calorimetry. In some cases, the expression or the activity of the gene implicated in fatty acid oxidation, weight loss, or fat loss is increased at least two-fold.

In some cases, the enhanced insulin sensitivity is evidenced by an increase in an expression or an activity of a gene implicated in insulin sensitivity. In some cases, the gene implicated in insulin sensitivity is selected from the group consisting of GLUT4, AdipoQ, and ChREBP. In some cases, the enhanced insulin sensitivity is evidenced by a decrease in fasting insulin, a decrease in homeostatic assessment of insulin resistance (HOMA_{IR}), a decrease in 60-minute glucose or insulin in a glucose tolerance test (GTT), or a decrease in the glucose or insulin area under the curve in a GTT. In some cases, the expression or the activity of the gene implicated in insulin sensitivity is increased at least two-fold.

In some cases, the administration of (a) and (b) is in an amount that synergistically enhances adipocyte beiging. In some cases, the administration of (a) and (b) is in an amount that synergistically enhances thermogenesis. In some cases, the administration of (a) and (b) is in an amount that synergistically enhances fatty acid oxidation, weight loss, or fat loss. In some cases, the administration of (a) and (b) is in an amount that synergistically enhances insulin sensitivity.

In yet another aspect, a composition is provided comprising a flavonoid and/or a flavonoid derivative in a range from 10 mg to 2000 mg and a sirtuin pathway activator in a range from 100 mg to 2000 mg. In some cases, the flavonoid and/or flavonoid derivative is provided in a range from 250 mg to 2000 mg. In some cases, the flavonoid and/or flavonoid derivative is provided in a range from 250 mg to 1000 mg. In some cases, the flavonoid and/or flavonoid derivative is provided in a range from 500 mg to 1000 mg. In some cases, the sirtuin pathway activator is provided in a range from about 100 mg to 1000 mg. In some cases, the sirtuin pathway activator is provided in a range from about 250 mg to 1000 mg.

In some cases, the flavonoid and/or flavonoid derivative comprises a flavanone. In some cases, the flavanone comprises blumeatin, butin, eriodictyol, hesperetin, hesperidin, homoeriodictyol, isosakuranetin, naringenin, naringin, pinocembrin, poncirin, sakuranetin, sakuranin, sterubin, and/or pinostrobin. In some cases, the sirtuin pathway activator comprises nicotinamide riboside, nicotinamide mononucleotide, nicotinic acid, or a nicotinic acid metabolite. In some cases, the composition is substantially free of nicotinamide. In some cases, the composition is substantially free of nicotinic acid metabolites. In some cases, the nicotinic acid metabolite is selected from the group consisting of nicotinyl CoA, nicotinuric acid, nicotinate mononucleotide, nicotinate adenine dinucleotide, and nicotinamide adenine dinucleotide. In some cases, the component (b) in the composition is nicotinamide riboside. In some cases, the component (a) in the composition is naringenin.

In some cases, the amount of sirtuin pathway activator is capable of achieving a serum level of the sirtuin pathway activator that is less than about 1 μM. In some cases, the amount of sirtuin pathway activator is capable of achieving a serum level of the sirtuin pathway activator that is about 10 μM. In some cases, the amount of sirtuin pathway activator is capable of achieving a serum level of the sirtuin pathway activator that is between about 1-100 μM.

In some cases, the composition is effective in increasing mitochondrial biogenesis, fatty acid oxidation, thermogenesis, weight loss, fat loss, insulin sensitivity, or adipocyte beiging in the subject by at least about 5%. In some cases, the amount of component (a) and (b) synergistically increases mitochondrial biogenesis, fatty acid oxidation, thermogenesis, weight loss, fat loss, insulin sensitivity, or adipocyte beiging in said subject when administered to the subject. In some cases, component (a) and component (b) synergistically enhances mitochondrial biogenesis, fatty acid oxidation, thermogenesis, weight loss, fat loss, insulin sensitivity, or adipocyte beiging of the subject, or an increase in activation of UCP1, GLUT4, ADIPOQ, CPT1b, ChREBP, ATGL, APMK or SIRT1, in the subject. In some cases, the amount of sirtuin pathway activator is insufficient to increase mitochondrial biogenesis, fatty acid oxidation, thermogenesis, weight loss, fat loss, insulin sensitivity, or adipocyte beiging in the absence of the flavonoid and/or one or more flavonoid derivatives.

In some cases, the composition is contained in a foodstuff.

In some cases, a portion of the flavonoid and/or one or more flavonoid derivatives is in a free form. In some cases, a portion of the flavonoid and/or one or more flavonoid derivatives is in a salt form.

In some cases, the composition is formulated for oral administration. In some cases, the composition is a tablet, a capsule, a pill, a granule, an emulsion, a gel, a plurality of beads encapsulated in a capsule, a powder, a suspension, a liquid, a semi-liquid, a semi-solid, a syrup, a slurry or a chewable form.

In some cases, component (a) and component (b) are separately packaged. In some cases, component (a) and component (b) are mixed.

In some cases, the one or more flavonoid derivatives are selected from the group consisting of naringin or 8-prenylnaringenin. In some cases, the composition does not contain nicotinamide.

In some cases, the composition further comprises one or more therapeutic agents that is capable of increasing mitochondrial biogenesis, fatty acid oxidation, thermogenesis, weight loss, fat loss, insulin sensitivity, or adipocyte beiging. In some cases, the one or more therapeutic agents is selected from the group consisting of orlistat, cetilistat, lorcaserin, sibutramine, rimonabant, metformin, exenatide, liraglutide, semaglutide, amylin, pramlinatide, phentermine, topiramate, bupropion, naltrexone, and amphetamine, and any combination thereof.

In some cases, the molar ratio of component (a) to (b) in said composition is greater than about 0.1.

In some cases, the composition is formulated in a unit dosage form.

In yet another aspect, a kit is provided comprising a multi-day supply of unit dosages of the composition and instructions directing the administration of said multi-day supply over a period of multiple days.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the change in UCP1 gene expression in hASC cells for various dosing combinations of naringenin and nicotinamide riboside.

FIG. 2 depicts the change in Glut4 gene expression in hASC cells for various dosing combinations of naringenin and nicotinamide riboside.

FIG. 3 depicts the change in CPT1b gene expression in hASC cells for various dosing combinations of naringenin and nicotinamide riboside.

FIG. 4 depicts the change in AdipoQ gene expression in hASC cells for various dosing combinations of naringenin and nicotinamide riboside.

FIG. 5 depicts the change in ChREBP gene expression in hASC cells for various dosing combinations of naringenin and nicotinamide riboside.

FIG. 6 depicts the change in ATGL gene expression in hASC cells for various dosing combinations of naringenin and nicotinamide riboside.

FIG. 7 depicts the change in PRDM16 gene expression in hASC cells for various dosing combinations of naringenin and nicotinamide riboside.

### DETAILED DESCRIPTION OF THE INVENTION

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “subject” or “individual” includes mammals. Non-limiting examples of mammals include humans and mice, including transgenic and non-transgenic mice. The methods described herein can be useful in both human therapeutics, pre-clinical, and veterinary applications. In some embodiments, the subject is a mammal, and in some embodiments, the subject is human. Other mammals include, and are not limited to, apes, chimpanzees, orangutans, monkeys; domesticated animals (pets) such as dogs, cats, guinea pigs, hamsters, mice, rats, rabbits, and ferrets; domesticated farm animals such as cows, buffalo, bison, horses, donkey, swine, sheep, and goats; or exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, pandas, giant pandas, hyena, seals, sea lions, and elephant seals.

The terms “administer”, “administered”, “administers” and “administering” are defined as the providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments of the subject application, oral routes of administering a composition can be preferred.

As used herein, “agent” or “biologically active agent” refers to a biological, pharmaceutical, or chemical compound or other moiety. Non-limiting examples include simple or complex organic or inorganic molecule, a peptide, a protein, a peptide nucleic acid (PNA), an oligonucleotide (including e.g., aptamer and polynucleotides), an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

The term “effective amount” or “therapeutically effective amount” refers to that amount of a compound described herein that is sufficient to affect the intended application including but not limited to disease or condition treatment, as defined below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or down regulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

The term “energy metabolism,” as used herein, refers to the transformation of energy that accompanies biochemical reactions in the body, including cellular metabolism and mitochondrial biogenesis. Energy metabolism can be quantified using the various measurements described herein, for example and without limitations, weight-loss, fat-loss or weight loss, insulin sensitivity, fatty acid oxidation, thermogenesis, adipocyte beiging, glucose utilization, triglyceride content, Sirt 1 activity or expression level, other sirtuin activity or expression level, AMPK activity or expression level, oxidative stress, Uncoupling Protein 1 (UCP1) expression level, and mitochondrial biomass.

The term “isolated”, as applied to the subject components, for example a PDE 5 inhibitor, including but not limited to nicotinic acid and/or nicotinamide riboside and/or nicotinamide mononucleotide and/or one or more nicotinic acid metabolites, naringenin and naringenin metabolites, and resveratrol, refers to a preparation of the substance devoid of at least some of the other components that can also be present where the substance or a similar substance naturally occurs or is initially obtained from. Thus, for example, an isolated substance can be prepared by using a purification technique to enrich it from a source mixture. Enrichment can be measured on an absolute basis, such as weight per volume of solution, or it can be measured in relation to a second, potentially interfering substance present in the source mixture. Increasing enrichment of the embodiments of this invention are increasingly more preferred. Thus, for example, a 2-fold enrichment is preferred, 10-fold enrichment is more preferred, 100-fold enrichment is more preferred, 1000-fold enrichment is even more preferred. A substance can also be provided in an isolated state by a process of artificial assembly, such as by chemical synthesis.

A “sub-therapeutic amount” of an agent, an activator or a therapy is an amount less than the effective amount of that agent, activator or therapy for an intended application, but when combined with an effective or sub-therapeutic amount of another agent or therapy can produce a desired result, due to, for example, synergy in the resulting efficacious effects, and/or reduced side effects.

A “synergistic” or “synergizing” effect can be such that the one or more effects of the combination compositions are greater than the one or more effects of each component alone, or they can be greater than the sum of the one or more effects of each component alone. The synergistic effect can be about, or greater than about 10, 20, 30, 50, 75, 100, 110, 120, 150, 200, 250, 350, or 500% or even more than the effect on a subject with one of the components alone, or the additive effects of each of the components when administered individually. The effect can be any of the measurable effects described herein.

The term “substantially free”, as used herein, refers to compositions that have less than about 10%, less than about 5%, less than about 1%, less than about 0.5%, less than 0.1% or even less of a specified component. For example, a composition that is substantially free of flavonoid derivatives or nicotinamides can have less than about 1% of the flavonoid derivatives or nicotinamides. The percentage can be determined as a percent of the total composition or a percent of a subset of the composition. For example, a composition that is substantially free of nicotinamides can have less than 1% of the nicotinamides as a percent of the total composition, or as a percent of the sirtuin pathway activator compounds in the composition. The percentages can be mass, molar, or volume percentages.

The terms “clinical significance” or “clinically significant” indicate behaviors and symptoms that are considered to be outside the range of normal, and are marked by distress and impairment of daily functioning. For example, a clinically significant cutaneous vasodilation would be a level sufficient to elicit patient complaint regarding discomfort secondary to acute vasodilatation, including flushing, itching and/or tingling. Levels of cutaneous vasodilation can also be measured by any methods known in the medical art, such as the methods including laser-Doppler flowmeter that are disclosed in Saumet J. L. et al., “Non-invasive measurement of skin blood flow: comparison between plethysmography, laser-Doppler flowmeter and heat thermal clearance method” Int. J. Microcirc. Clin. Exp. 1986; 5:73-83. A clinically significant level of cutaneous vasodilation can also be a level that is statistically significant. A clinically significant level of cutaneous vasodilation can also be a level that is not statistically significant.

The terms “lipid content” or “lipid level” refer to the content or level of lipid or lipoprotein molecules measured inside of a subject. It can be the concentration of the lipid molecules in a circulating bloodstream, or a total quantity of body fat. The lipid or lipoprotein molecules can include triglyceride, cholesterol, LDL, or HDL.

### Compositions

The invention provides for compositions that can increase or modulate the output of one or more pathways involved in adipocyte beiging, thermogenesis, fatty acid oxidation, fat loss or weight loss, and insulin sensitivity. These pathways, such as the sirtuin pathway, include, without limitation, signaling molecules such as, Sirt1, Sirt3, and AMPK. The output of the pathway can be determined by the expression level and/or the activity of the pathway and/or a physiological effect. In some embodiments, activation of a pathway includes stimulation of PGC1-α and/or subsequent stimulation of fatty acid oxidation and/or mitochondrial biogenesis. In general, a sirtuin pathway activator is a compound that activates or increases one or more components of a sirtuin pathway. An increase or activation of a sirtuin pathway can be observed by an increase in the activity of a pathway component protein. For example, the protein can be Sirt1, PGC1-α, AMPK, Epac1, Adenylyl cyclase, Sirt3, or any other proteins and their respective associated proteins along the signaling pathway (Park et. al., “Resveratrol Ameliorates Aging-Related Metabolic Phenotypes by Inhibiting cAMP Phosphodiesterases,” Cell 148, 421-433 Feb. 3, 2012). Non-limiting examples of physiological effects that can serve as measures of relevant pathway output include mitochondrial biogenesis, fatty acid oxidation, fat loss or weight loss, heat production, visceral adipose tissue loss, insulin sensitivity, and browning of fat cells. Examples of indicia of browning of fat cells include, without limitation, increased fatty acid oxidation, and expression of one or more brown-fat-selective genes (e.g. Ucp1, Cidea, Prdm16, and Ndufs1). In some embodiments, changes in one or more physiological effects that can serve as measures of pathway output (e.g., sirtuin pathway output) are induced by administering a composition of the invention.

An increase in mitochondrial biogenesis can be evidenced by an increase in the formation of new mitochondria and/or by an increase in mitochondrial functions, such as increased fatty acid oxidation, increased heat generation, increased insulin sensitivity, increased in glucose uptake, increased in vasodilation, decreased in weight, decreased in adipose volume, and decreased inflammatory response or markers in a subject.

The compositions can be combination compositions which may include one or more synergistic components. In some embodiments, the synergistic effect of the combination compositions can allow for reduced dosing amounts, leading to reduced side effects to the subject and reduced cost of treatment. In other embodiments, the synergistic effect can allow for results that are not achievable through any other conventional treatments. The subject combination compositions provide a significant improvement in the regulation of energy metabolism. In some embodiments, the composition may comprise a synergistic combination of a flavonoid component and a sirtuin pathway activator.

In some embodiments, the compositions can be combination compositions of one or more flavonoid compounds and/or metabolites thereof and a sirtuin-pathway activator that can have one or more characteristics. The combination compositions (a) can have a synergistic effect in increasing the sirtuin-pathway output, (b) increase sirtuin-pathway output by at least or at least about 1, 2, 5, 7, 10, or 20 fold, (c) have a molar ratio of flavonoid compounds and/or metabolites thereof to sirtuin-pathway output that is greater than or greater than about 3, (d) be formulated as a unit dosage for oral ingestion, where the sirtuin-pathway activator is a substantially homogeneous population of active or inactive compounds, and (e) can have a synergistic effect and further comprise a food carrier. Any of the compositions described herein can have one or more of these characteristics.

In some embodiments, the present invention provides a composition comprising (a) one or more types of flavonoids or derivatives thereof and (b) a sirtuin-pathway activator present in a sub-therapeutic amount, wherein the composition is synergistically effective in increasing the sirtuin-pathway output by at least or at least about 5, 10, 50, 100, 200, 500 or more fold as compared to that of component (b) when it being used alone.

The subject compositions comprise a combination of (i) flavonoids (e.g., flavanones such as naringenin) or derivatives thereof, and (ii) a sirtuin pathway activator such as nicotinamide riboside, nicotinamide mononucleotide, nicotinic acid, or one or more other nicotinic acid metabolites. The composition can be used to treat obesity and related metabolic disorders. The compositions can further comprise resveratrol or one or more therapeutic agents that is capable of altering cellular energy metabolism. The combination of these components can be useful for increasing the rate of fatty acid oxidation in energy-storing cells or decreasing the rate of fatty acid storage in energy-storing cells. In some embodiments, the components are formulated to provide a synergistic effect, including but not limited to further reduction of the fat content or reduction in dosing amounts leading to reduced side effects to the subject. The combination can be particularly effective in lowering the lipid content of cells while causing a reduced degree of side effects such as nausea, fatigue, headaches, and cutaneous vasodilation in a subject as compared to a dose of a sirtuin pathway activator alone that has the same effectiveness as the composition in boosting energy metabolism. The amount of sirtuin pathway activator such as nicotinamide riboside, nicotinamide mononucleotide, nicotinic acid, or one or more nicotinic acid metabolites in the composition can be a sub-therapeutic amount in the absence of the flavonoids (e.g., flavanones such as naringenin) or derivatives thereof.

In one embodiment, the subject composition comprises flavonoids (e.g., flavanones such as naringenin) or derivatives thereof, and a sirtuin pathway activator such as nicotinamide riboside, nicotinamide mononucleotide, nicotinic acid, and/or other nicotinic acid metabolites, wherein the composition produces a serum concentration of at least about 5 μM and/or at least about 0.5 μM of the one or more flavonoids (e.g., flavanones such as naringenin) or derivatives thereof, and further wherein the composition produces a serum concentration of at least about 15 μM of the sirtuin pathway activator.

In yet another embodiment, the subject composition comprises flavonoids (e.g., flavanones such as naringenin) or derivatives thereof; and an amount of a sirtuin pathway activator such as nicotinamide riboside, nicotinamide mononucleotide, nicotinic acid, or nicotinic acid metabolites, wherein the composition produces a serum concentration of at least about 5 μM and/or at least about 0.5 μM of the flavonoid (e.g., flavanones such as naringenin) or derivatives thereof, and further wherein the amount sirtuin pathway activator is insufficient to demonstrate a therapeutic effect such as increasing fatty acid oxidation in the absence of the flavonoid (e.g., flavanones such as naringenin) or derivatives thereof. In some embodiments, the amount of the sirtuin pathway activator is sub-therapeutic when administered without a flavonoid (e.g., flavanones such as naringenin) or derivatives thereof.

In still yet another embodiment, the subject composition comprises a flavonoid (e.g., flavanones such as naringenin) or derivatives thereof; and a sirtuin pathway activator, wherein the composition is effective in increasing fatty acid oxidation in a subject in need thereof while causing a reduced degree of side effects, such as nausea, fatigue, headaches, and cutaneous vasodilation in the subject as compared to a dose of sirtuin pathway activator alone that has the same effectiveness as the composition in increasing fatty acid oxidation. In some embodiments, the composition is effective in increasing fatty acid oxidation in a subject in need thereof without causing a clinically significant side effect, such as cutaneous vasodilation.

In another embodiment, the subject composition comprises (a) naringenin and/or one or more naringenin metabolites; and (b) nicotinamide riboside and/or nicotinamide mononucleotide and/or nicotinic acid and/or nicotinic acid metabolites, wherein the mass ratio of (a) to (b) is at least or at least about 0.1, 0.2, 0.3, 0.4, 0.5, 0.6, 0.7, 0.8, 0.9, 1, 2, 3, 4, 5, 6, 7, 8, 9, or 10 or more, and wherein the composition comprises at least or at least about 1 mg of the sirtuin pathway activator. As described herein, a dosing of at least or at least about 1 mg, 10 mg, 50 mg, 100 mg, or more than 100 mg of nicotinamide riboside and/or nicotinamide mononucleotide and/or nicotinic acid and/or nicotinic acid metabolites can provide a sub-therapeutic dosing that can be effective when combined with a sufficient mass ratio of naringenin or naringenin metabolite.

In some embodiments, the subject composition comprises (a) a flavonoid (e.g., flavanones such as naringenin) or derivatives thereof; and (b) nicotinamide riboside, nicotinamide mononucleotide, nicotinic acid, or nicotinic acid metabolites, wherein component (a) and component (b) have synergistic effects. The synergistic effects can be synergistically enhancing a decrease in weight gain of the subject, a decrease in lipid content, a decrease in LDL level, an increase in HDL level, a decrease in cholesterol level, a decrease in triglyceride level, an increase in thermogenesis, an increase in insulin sensitivity, an increase in fat oxidation of the subject, an increase in fat cell browning, or an increase in activation of Sirt1 in the subject.

In one aspect of the invention, the subject composition comprises (a) a flavonoid (e.g., flavanones such as naringenin) or derivatives thereof; and (b nicotinamide riboside, nicotinamide mononucleotide, nicotinic acid, or nicotinic acid metabolites. The composition can further comprise at least about 0.01, 0.05, 0.1, 0.5, 1, 10, 50, 100, 250, or 500 μg of resveratrol.

### Sirtuin Pathway Activating Compounds

The invention provides for compositions that include sirtuin pathway activators such as nicotinamide riboside, nicotinamide mononucleotide, nicotinic acid, or nicotinic acid metabolites. The sirtuin pathway activators can be used in free form. The term “free,” as used herein in reference to a component, indicates that the component is not incorporated into a larger molecular complex. The sirtuin pathway activators can be in a salt form.

In some embodiments, the compositions can be substantially free of nicotinamide and/or nicotinic acid and/or nicotinamide metabolites. The nicotinamide and/or nicotinic acid and/or nicotinamide metabolites can counteract the effects of nicotinamide riboside and/or nicotinamide mononucleotide and/or nicotinic acid and/or nicotinic acid metabolites. Nicotinamide can be harmful to the liver in high doses (as disclosed in http://www.livestrong.com/article/448906-therapeutic-levels-of-niacin-to-lower-cholesterol-levels/#ixzz2NO3KhDZu). The mass or molar amount of nicotinamide and/or nicotinamide metabolites can be less than or less than about 0.01, 0.1, 0.5, 1, 2, 5, or 10% of the total composition. The mass or molar amount of nicotinic acid and/or nicotinamide and/or nicotinamide metabolites can be less than or less than about 0.01, 0.1, 0.5, 1, 2, 5, or 10% of the total composition.

Without being limited to theory, ingestion of a sirtuin pathway activator, such as nicotinamide riboside and/or nicotinamide mononucleotide and/or nicotinic acid, and/or nicotinic acid metabolites, can increase mitochondrial biogenesis, adipocyte beiging, fatty acid oxidation, fat loss or weight loss, thermogenesis, insulin sensitivity, or stimulate sirtuin signaling, including increase activation of Sirt1 and Sirt3. The ingestion of nicotinamide riboside, nicotinamide mononucleotide, nicotinic acid, or nicotinic acid metabolites can also lower lipid content, lower triglyceride level, lower LDL level, lower total cholesterol level, or increase HDL level. In some embodiments, any of the compositions described herein can include salts, derivatives, metabolites, catabolites, anabolites, precursors, and analogs of nicotinic acid. For example, the metabolites can include nicotinyl CoA, nicotinuric acid, nicotinate mononucleotide, nicotinate adenine dinucleotide, or nicotinamide adenine dinucleotide. In some embodiments, the compositions cannot comprise nicotinamide. In some embodiments, the compositions comprise nicotinamide. In some embodiments, the compositions can be substantially free of nicotinic acid metabolites.

### Flavonoids and Flavonoid Derivatives

The invention provides for compositions that include flavonoids and flavonoid derivatives. The flavonoids may include bioflavonoids, isoflavonoids, or neoflavonoids. A composition of the present invention may comprise one or more flavonoids selected from the flavonoid subgroups of flavones, flavonols, 3-hydroxyflavones, flavanones, flavanonol, 3-hydroxyflavanones, 2,3-dihydroflavonols, and flavans. In some cases, a composition may comprise one or more flavanones, including blumeatin, butin, eriodictyol, hesperetin, hesperidin, homoeriodictyol, isosakuranetin, naringenin, naringin, pinocembrin, poncirin, sakuranetin, sakuranin, sterubin, and/or pinostrobin. The flavonoids compound may be derived from a biological source (e.g., plant, microbe, or engineered microbe) or produced synthetically. The flavonoid compound or derivatives thereof can be used in free form. The term “free,” as used herein in reference to a component, indicates that the component is not incorporated into a larger molecular complex. The flavonoid compound or derivative thereof can be in a salt form.

Without being limited to theory, ingestion of flavonoids, such as naringenin, can stimulate certain metabolic pathways (e.g., AMPK signaling), which can favorably modulate inflammatory cytokine patterns. In some embodiments, any of the compositions described herein can include salts, derivatives, metabolites, catabolites, anabolites, precursors, and analogs of flavonoids. For example, the related compounds of naringenin can include naringin and 8-prenylnaringenin.

In some embodiments, the compositions can be substantially free of one or more, or all non-naringenin flavonoids. For example, the compositions can be free of blumeatin, butin, eriodictyol, hesperetin, hesperidin, homoeriodictyol, isosakuranetin, naringin, pinocembrin, poncirin, sakuranetin, sakuranin, sterubin, and/or pinostrobin.

### Therapeutic Agents

The subject compositions can further include one or more pharmaceutically active agents or therapeutic agents other than sirtuin pathway activators. The therapeutic agents or pharmaceutically active agents can be any agent that is known in the art. For example, the combination compositions can further comprise a pharmaceutically active anti-obesity agent, or a dietary supplement that also effects on energy metabolism. The anti-obesity agent can be an oral agent or injectable agent. The anti-obesity agents can be in a sub-therapeutic amount in increasing mitochondrial biogenesis, fatty acid metabolism, increasing thermogenesis, increasing adipocyte beiging, fat loss or weight loss, or increasing insulin sensitivity. The types of the anti-obesity agents known in the art can include, but are not limited to, orlistat, cetilistat, lorcaserin, sibutramine, rimonabant, metformin, exenatide, liraglutide, semaglutide, amylin, pramlinatide, phentermine, topiramate, bupropion, naltrexone, and amphetamine. These examples are provided for discussion purposes only, and are intended to demonstrate the broad scope of applicability of the invention to a wide variety of drugs. It is not meant to limit the scope of the invention in any way.

The combination compositions can further include one or more other pharmaceutically active agents. Examples of therapeutically active agents include ibuprofen, aldoril, and gemfebrozil, verapamil, maxzide, diclofenac and metrolol, maproltiline, triazolam and minoxidil. For example, the combination compositions can comprise a pharmaceutically active anti-diabetic agent, weight loss agent, or calcium regulation agent. U.S. Pat. No. 7,109,198 and U.S. Patent Application No. 20090142336 describe a variety of pharmaceutically active agents or therapeutically active agents suitable for inclusion in a combination composition described herein. Examples of anti-diabetic agents include biguanides (such as metformin), thiazoladinediones and meglitinides (such as repaglinide, pioglitazone, and rosiglitazone), alpha glucosidease inhibitors (such as acarbose), sulfonylureas (such as tolbutamide, acetohexamide, tolazamide, chlorpropamide, glipizide, glyburide, glimepiride, gliclazide), incretins, ergot alkaloids (such as bromocriptine), and DPP inhibitors (such as sitagliptin, vildagliptin, saxagliptin, lingliptin, dutogliptin, gemigliptin, alogliptin, and berberine). The anti-diabetic agent can be an oral anti-diabetic agent. The anti-diabetic agent can also be injectable anti-diabetic drugs, including insulin, amylin analogues (such as pramlintide), and incretin mimetics (such as exenatide and liraglutide). Examples of anti-obesity therapeutic agents include lipase inhibitors (such as Orlistat), dopaminergic, noradrenergic, and serotoninergic compounds, cannabinoid receptor antagonists (such as rimonabant), exenatide, pramlintide, and CNS agents (such as topimerate). These examples are provided for discussion purposes only, and are intended to demonstrate the broad scope of applicability of the invention to a wide variety of drugs. It is not meant to limit the scope of the invention in any way.

In some embodiments, one or more components described herein, such as resveratrol, leucine, HMB, and KIC can be combined with two or more pharmaceutically active agents. For example, a sirtuin pathway activator can be combined with glipizide and metformin, glyburide and metformin, pioglitazone and glimepiride, pioglitazone and metformin, repaglinide and metformin, rosiglitazone and glimepiride, rosiglitazone and metformin, or sitagliptin and metformin.

The subject composition can further comprise one or more therapeutic agents that are herbs and/or supplements. The herbs and/or supplements can have therapeutic effects that are unproven scientifically. The examples of the herbs and/or the supplements can be, but are not limited to: Acai, Alfalfa, Aloe, Aloe Vera, Aristolochic Acids, Asian Ginseng, Astragalus, Bacillus coagulans, Belladonna, Beta-carotene, Bifidobacteria, Bilberry, Bilberry, Biotin, Bitter Orange, Black Cohosh, Black Cohosh, Black psyllium, Black tea, Bladderwrack, Blessed thistle, Blond psyllium, Blueberry, Blue-green algae, Boron, Bromelain, Butterbur, Calcium, Calendula, Cancell/Cantron/Protocel, Cartilage (Bovine and Shark), Cassia cinnamon, Cat's Claw, Chamomile, Chasteberry, Chondroitin sulfate, Chromium, Cinnamon, Clove, Coenzyme Q-10, Colloidal Silver Products, Cranberry, Creatine, Dandelion, Dandelion, Devil's claw, DHEA, Dong quai, Echinacea, Ephedra, Essiac/Flor-Essence, Eucalyptus, European Elder (Elderberry), European Mistletoe, Evening Primrose Oil, Fenugreek, Feverfew, Fish oil, Flaxseed, Flaxseed oil, Folate, Folic acid, Garlic, Ginger, Gingko, Ginseng, Glucosamine hydrochloride, Glucosamine sulfate, Goldenseal, Grape Seed Extract, Green Tea, Hawthorn, Hoodia, Horse Chestnut, Horsetail, Hydrazine Sulfate, Iodine, Iron, Kava, Lactobacillus, Laetrile/Amygdalin, L-arginine, Lavender, Licorice, Lycium, Lycopene, Magnesium, Manganese, Melatonin, Milk Thistle, Mistletoe Extracts, Noni, Oral Probiotics, Pantothenic acid (Vitamin B5), Passionflower, PC-SPES, Pennyroyal, Peppermint, Phosphate salts, Pomegranate, Propolis, Pycnogenol, Pyridoxine (Vitamin B6), Red Clover, Red yeast, Riboflavin (Vitamin B2), Roman chamomile, Saccharomyces boulardii, S-Adenosyl-L-Methionine (SAMe), Sage, Saw Palmetto, Selected Vegetables/Sun's Soup, Selenium, Senna, Soy, St. John's Wort, sweet orange essence, Tea Tree Oil, Thiamine (Vitamin B1), Thunder God Vine, Turmeric, Valerian, Vitamin A, Vitamin B12, Vitamin C, Vitamin D, Vitamin E, Vitamin K, Wild yam, Yohimbe, Zinc or 5-HTP.

The amount of pharmaceutical agent, or any other component used in a combination composition described herein, can be a used in an amount that is sub-therapeutic. In some embodiments, using sub-therapeutic amounts of an agent or component can reduce the side-effects of the agent. Use of sub-therapeutic amounts can still be effective, particularly when used in synergy with other agents or components.

A sub-therapeutic amount of the agent or component can be such that it is an amount below which would be considered therapeutic. For example, FDA guidelines can suggest a specified level of dosing to treat a particular condition, and a sub-therapeutic amount would be any level that is below the FDA suggested dosing level. The sub-therapeutic amount can be or can be about 1, 5, 10, 15, 20, 25, 30, 35, 40, 45, 50, 55, 60, 65, 70, 75, 80, 85, 90, or 95% less than the amount that is considered to be a therapeutic amount. The therapeutic amount can be assessed for individual subjects, or for groups of subjects. The group of subjects can be all potential subjects, or subjects having a particular characteristic such as age, weight, race, gender, or physical activity level.

In the case of nicotinamide riboside, nicotinamide mononucleotide, nicotinic acid, or nicotinic acid metabolites administered alone to increase fatty acid metabolism, the current physician suggested starting dose is 1000-3000 mg daily for nicotinic acid, with subject specific dosing having a range of 1 mg to a maximum of 1000 mg daily when administered with a flavonoid or a derivative thereof. The particular dosing for a subject can be determined by a clinician by titrating the dose and measuring the therapeutic response. The therapeutic dosing level can be determined by measuring any fatty acid metabolism biomarkers without causing clinically significant cutaneous vasodilation. A sub-therapeutic amount can be any level that would be below the recommended dosing of nicotinamide riboside, nicotinamide mononucleotide, or nicotinic acid. For example, if a subject's therapeutic dosing level is determined to be 700 mg daily, a dose of 600 mg would be a sub-therapeutic amount. Alternatively, a sub-therapeutic amount can be determined relative to a group of subjects rather than an individual subject. For example, if the average therapeutic amount of nicotinic acid, nicotinamide riboside, nicotinamide mononucleotide, or nicotinic acid metabolites for subjects with weights over 300 lbs is 2000 mg, then a sub-therapeutic amount can be any amount below 2000 mg. In some embodiments, the dosing can be recommended by a healthcare provider including, but not limited to a patient's physician, nurse, nutritionist, pharmacist, or other health care professional. A health care professional can include a person or entity that is associated with the health care system. Examples of health care professionals can include surgeons, dentists, audiologists, speech pathologists, physicians (including general practitioners and specialists), physician assistants, nurses, midwives, pharmaconomists/pharmacists, dietitians, therapists, psychologists, physical therapists, phlebotomists, occupational therapists, optometrists, chiropractors, clinical officers, emergency medical technicians, paramedics, medical laboratory technicians, radiographers, medical prosthetic technicians social workers, and a wide variety of other human resources trained to provide some type of health care service.

In the case of nicotinamide riboside, nicotinamide mononucleotide, nicotinic acid, or nicotinic acid metabolites, the therapeutically effective level of the nicotinamide riboside, nicotinamide mononucleotide, nicotinic acid, or nicotinic acid metabolites can be a circulating level between about 1-100 nM. A sub-therapeutic level of the of nicotinamide riboside, nicotinamide mononucleotide, nicotinic acid, or nicotinic acid metabolites, by itself or in any combination, can be any circulating level at least, at least about, less than, less than about, more than, or more than about 1, 2.5, 5, 10, 15, 20, 25, 30, 35, 40, 45, 50, 60, 70, 80, 90 or 100 nM. The sub-therapeutic level of the of nicotinamide riboside, nicotinamide mononucleotide, nicotinic acid, or nicotinic acid metabolites, in a subject composition formulated for administration can be, can be about, can be less than, can be less than about, can be at least, or can be at least about 1, 5, 10, 20, 25, 30, 40, 50, 60, 70, 80, 90, 100, 125, 150, 175, 200, 250, 300, 350, 400, 450, 500, 600, 700, 750, 800, 900, 1000, 1100, 1200, 1300, 1400, 1500, 1600, 1700, 1800, 1900, or 2000 mg of the of nicotinamide riboside, nicotinamide mononucleotide, nicotinic acid, or nicotinic acid metabolites.

Any of the components described herein, including a flavonoid (e.g., naringenin), a sirtuin pathway activator (e.g., nicotinamide riboside), and a therapeutic agent (e.g., resveratrol) can be used in a subject composition in free form, isolated form, purified from a natural source, and/or purified or prepared from a synthetic source. The natural source can be an animal source or plant source. The components can be pure to at least or at least about 95, 97, 99, 99.5, 99.9, 99.99, or 99.999%.

### Dosing Amounts

The invention provides for compositions that are combinations of isolated components, such as flavonoids or derivatives of flavonoids, sirtuin pathway activators, and/or therapeutic agents, that have been isolated from one or more sources. The invention provides for compositions that are enriched in flavonoids or flavonoid derivatives, sirtuin pathway activators and/or therapeutic agents. The components can be isolated from natural sources or created from synthetic sources and then enriched to increase the purity of the components. Additionally, flavonoids or flavonoid derivatives can be isolated from a natural source and then enriched by one or more separations. The isolated and enriched components, such as naringenin, can then be combined and formulated for administration to a subject.

In some embodiments, a composition comprises an amount of sirtuin pathway activator. The amount of sirtuin pathway activator, such as nicotinamide riboside, nicotinamide mononucleotide, nicotinic acid, or nicotinic acid metabolites can be a subtherapeutic amount, and/or an amount that is synergistic with one or more other compounds in the composition or one or more of the compounds administered simultaneously or in close temporal proximity with the composition. In some embodiments, the sirtuin pathway activator is administered in a low dose, a medium dose, or a high dose, which describes the relationship between two doses, and generally do not define any particular dose range. The compositions can be administered to a subject such that the subject is administered a selected total daily dose of the composition. The total daily dose can be determined by the sum of doses administered over a 24 hour period.

A dose, which can be a unit dose, can comprise, can comprise about, can comprise more than, can comprise more than about, can comprise less than, or can comprise less than about 10, 20, 30, 40, 50, 60, 70, 80, 90, 100, 110, 120, 130, 140, 150, 160, 170, 180, 190, 200, 250, 400, 500, 550, 600, 700, 800, 900, 1000, 1100, 1250, 1300, 1350, 1400, 1500, 1600, 1700, 1800, 1900, or 2000, 2100, 2200, 2300, 2400, 2500, 2600, 2700, 2800, 2900, or 3000 or more mg of a flavonoid or flavonoid derivative. The flavonoid or flavonoid derivative can be a free flavonoid or flavonoid derivative. In some embodiments, a unit dose can comprise or can comprise at least about 1000 mg of free flavonoid or flavonoid derivative. The composition can comprise between or between about 10-2000, 200-2000, 500-2000, 10-1000, 500-1000, 10-500, 250-500, or 10-250 mg of flavonoid or flavonoid derivative. A dose, which can be a unit dose, can comprise, can comprise about, can comprise more than, can comprise more than about, can comprise less than, or can comprise less than about 10, 15, 20, 25, 30, 35, 40, 45, 50, 100, 200, 250, 400, 500, 550, 600, 700, 800, 900, 1000, 1250, 1300, 1350, 1400, 1450, 1500, 1600, 1700, 1800, 1900, 2000, 2100, 2200, 2300, 2400, 2500, 2600, 2700, 2800, 2900, or 3000 or more mg of a flavonoid or flavonoid derivative metabolite, such as naringenin. The flavonoid derivative can be a free flavonoid derivative metabolite. The composition can comprise between about 10-2000, 200-2000, 500-2000, 10-1000, 500-1000, 10-500, 250-500, or 10-250 mg of the flavonoid derivative or metabolite, such as 8-prenylnaringenin or naringin. In some embodiments, a unit dose can comprise at least or at least about 100, 200, 300, 400, 500, 600, 700, 800, 900, 1000, or 1500 mg of free flavonoid derivative. The amount of flavonoid or flavonoid derivative as described herein can be administered daily or simultaneously. The amount as described herein can be administered in one dose or separately in multiple doses daily.

In some embodiments, a daily dose of flavonoid can be, can be about, can be less than, can be less than about, can be more than, or can be more than about 0.25-3 or 0.5-3.0 g/day (e.g. 0.5, 0.75, 1, 1.25, 1.5, 1.75, 2, 2.5, 3, or more g/day). A daily dose of flavonoid derivative can be, can be about, can be less than, can be less than about, can be more than, or can be more than about 0.20-3.0 g/day (e.g. 0.2, 0.4, 0.5, 0.75, 1, 1.5, 2, 2.5, 3, or more g/day). A daily dose of naringenin can be, can be about, can be less than, can be less than about, can be more than, or can be more than about 0.2-3.0 g/day (e.g. 0.2, 0.4, 0.5, 0.75, 1, 1.25, 1.5, 1.75, 2, 2.5, 3, or more g/day).

The dose of flavonoid or flavonoid derivative, can be a therapeutic dose. The dose of flavonoid or flavonoid derivative can be a sub-therapeutic dose. A sub-therapeutic dose of flavonoid or flavonoid derivative can be, can be about, can be less than, can be less than about, can be more than, or can be more than about 0.25-3.0 g (e.g. 0.25, 0.5, 0.75, 1, 1.25, 1.5, 1.75, 2, 2.5, 3, or more g). A sub-therapeutic dose of flavonoid or flavonoid derivative can be, can be about, can be less than, can be less than about, can be more than, or can be more than about 0.25-3.0 g/day (e.g. 0.25, 0.5, 0.75, 1, 1.25, 1.5, 1.75, 2, 2.5, 3, or more g/day). In some embodiments, the compositions comprises less than 3.0 g daily dosage of flavonoid or flavonoid derivative. A sub-therapeutic dose of naringenin can be, can be about, can be less than, can be less than about, can be more than, or can be more than about 0.05-3.0 g (e.g. 0.05, 0.1, 0.2, 0.4, 0.5, 0.75, 1, 1.5, 2, 2.5, 3, or more g).

A dose, which can be a unit dose, can comprise a sirtuin pathway activator, that can be, can be about, can be less than, can be less than about, can be more than, or can be more than about 10, 20, 25, 30, 35, 40, 45, 50, 60, 70, 80, 90, 100, 200, 250, 400, 500, 800, 1000, 1100, 1200, 1300, 1400, 1500, 1600, 1700, 1800, 1900, 2000, 2100, 2200, 2300, 2400, 2500, 2600, 2700, 2800, 2900, or 3000 mg of the sirtuin pathway activator. The composition can comprise between or between about 10-2000, 200-2000, 500-2000, 10-1000, 500-1000, 10-500, 250-500, or 10-250 of the sirtuin pathway activator. In some embodiments, a unit dose can comprise at least or at least about 1 mg of sirtuin pathway activator. In some embodiments, a unit dose can comprise less than or less than about 2000 mg of sirtuin pathway activator. The dosage can be adjusted for the intended subject administered. For example, a dose that is suitable for a canine can be less than the dose that is suitable for a human. The amount of sirtuin pathway activator as described herein can be administered daily or simultaneously. The amount as described herein can be administered in one dose or separately in multiple doses daily. The sirtuin pathway activator may comprise nicotinamide riboside, nicotinamide mononucleotide, nicotinic acid, or a nicotinic acid metabolite.

In some embodiments, the composition comprises at least two of nicotinamide riboside, nicotinamide mononucleotide, nicotinic acid, and nicotinic acid metabolites, and the total amount of the at least two compounds can be, can be about, can be more than, can be more than about, can be less than, or can be less than about 50, 60, 80, 100, 200, 250, 400, 500, 600, 800, 900, 1000, 1500, or 2000 mg.

In other embodiments, a daily dose of sirtuin pathway activator can be, can be about, can be more than, can be more than about, can be less than, or can be less than about 0.0001 mg/kg (mg of sirtuin pathway activator/kg of the subject receiving the dose), 0.005 mg/kg, 0.01 mg/kg, 0.5 mg/kg, 1 mg/kg, 2.5 mg/kg, 5 mg/kg, 7.5 mg/kg, 10 mg/kg, 12.5 mg/kg, 15 mg/kg, 20 mg/kg, 25 mg/kg, 50 mg/kg, 75 mg/kg, 100 mg/kg, or more.

A dose, which can be a unit dose, can comprise can be, can be about, can be more than, can be more than about, can be less than, or can be less than about 1, 5, 10, 25, 35, 50, 51, 75, 100, 150, 200, 250, 300, 350, 400, 450, 500, or more mg of resveratrol. The composition can comprise between or between about 5-500, 30-250, or 35-100 mg of resveratrol. In some embodiments, a unit dose can comprise at least or at least about 35 mg of resveratrol. The amount of resveratrol as described herein can be administered daily or simultaneously. The amount as described herein can be administered in one dose or separately in multiple doses daily.

A daily low dose of resveratrol can comprise, can comprise about, can comprise less than, can comprise less than about, can comprise more than, or can comprise more than about 0.5 mg/kg (mg of resveratrol/kg of the subject receiving the dose), 1 mg/kg, 2.5 mg/kg, 5 mg/kg, 7.5 mg/kg, 10 mg/kg, 12.5 mg/kg, 15 mg/kg, or more; a daily medium dose of resveratrol can comprise, can comprise about, can comprise less than, can comprise less than about, can comprise more than, or can comprise more than about 20 mg/kg, 25 mg/kg, 50 mg/kg, or more; and a daily high dose of resveratrol can comprise, can comprise about, can comprise less than, can comprise less than about, can comprise more than, or can comprise more than about 50 mg/kg, 75 mg/kg, 100 mg/kg, 125 mg/kg, 150 mg/kg, 175 mg/kg, 200 mg/kg, or more. The dosing range as defined to low, medium or high can be dependent on the subject receiving the dose and vary from subject to subject.

In some embodiments of the invention, the combination compositions can have a specified ratio of flavonoid or flavonoid derivative to sirtuin pathway activator, or flavonoid or flavonoid derivative to nicotinamide riboside, nicotinamide mononucleotide, nicotinic acid, or nicotinic acid metabolite. The specified ratio can provide for effective and/or synergistic treatment of obesity-linked conditions, which, for example, can be measured as a reduction in total lipid content, reduction in cholesterol level, reduction in triglyceride level, reduction in LDL level, reduction in body weight, and/or increase in HDL level, adipocyte beiging, thermogenesis, insulin sensitivity, fat loss or weight loss, or fatty acid metabolism. The ratio of flavonoid or flavonoid derivative to a sirtuin pathway activator can be a mass ratio, a molar ratio, or a volume ratio.

In some embodiments, a composition can comprise (a) a flavonoid or flavonoid derivative and (b) a sirtuin pathway activator (e.g., nicotinamide riboside, nicotinamide mononucleotide, nicotinic acid, or a nicotinic acid metabolite), where the mass ratio of (a) to (b) can be, can be about, can be more than, can be more than about, can be less than, or can be less than about 0.1, 0.2, 0.3, 0.4, 0.5, 0.6, 0.7, 0.8, 0.9, 1, 2, 5, 10, 15, 20, 25, 50, 75, 100, 200, 300, 350, 400, 450, 500, 550, 600, 650, 700, 750, or 800. In some embodiments, the mass ratio of (a) to (b) is at least or at least about 0.2. In some embodiments, the mass ratio of (a) to (b) is at least about 0.5. The composition can also comprise a minimal amount of sirtuin pathway activator, such as 5, 10 or 50 mg of the sirtuin pathway activator or a range of sirtuin pathway activator amount, such as 5-250 mg of nicotinamide riboside and/or nicotinamide mononucleotide and/or nicotinic acid metabolites. In some embodiments, the dosing of flavonoid or flavonoid derivative, sirtuin pathway activator, and therapeutic agent can be designed to achieve a specified physiological concentration or circulating level of flavonoid or flavonoid derivative, sirtuin pathway activator and/or therapeutic agent. The physiological concentration can be a circulating level as measured in the serum or blood stream of a subject. The subject can be a human or an animal. A selected dosing can be altered based on the characteristics of the subject, such as weight, rate of energy metabolism, genetics, ethnicity, height, or any other characteristic.

In some embodiments, a selected dose of a composition can be administered to a subject such that the subject achieves a desired circulating level of the composition. The desired circulating level of a component can be either a therapeutically effective level or a sub-therapeutic level.

The amount of flavonoid or flavonoid derivative in a unit dose can be such that the circulating level of flavonoid or flavonoid derivative in a subject is, is about, is greater than, or is greater than about 1 μM, 5 μM, 10 μM, 25 μM, 50 μM, 0.10 mM, 0.25 mM, 0.5 mM, 0.75 mM, or 1 mM. A dosing of about 1,125 mg flavonoid or flavonoid derivative (e.g., free naringenin), can achieve a circulating level of flavonoid or flavonoid derivative in a subject that is or is about 0.5 mM. A dosing of or of about 300 mg flavonoid or flavonoid derivative (e.g., free naringenin), can achieve a circulating level of flavonoid or flavonoid derivative in a subject that is or is about 0.25 mM.

The desired circulating level of the composition can be, can be about, can be more than, can be more than about, can be less than, or can be less than about 0.1 nM, 0.25 nM, 0.5 nM, 0.75 nM, 1 nM, 10 nM, 20 nM, 40 nM, 60 nM, 80 nM, 100 nM, 120 nM, 200 nM, 400 nM, 500 nM, 1 μM, 1.5 μM, 2 μM, 2.5 μM, or 3 μM, 4 μM, 5 μM, 10 μM, 25 μM, 50 μM, 100 μM or more of the sirtuin pathway activator. The therapeutically effective level of sirtuin pathway activator can be between or can be between about 1-111 μM, which corresponds to about 1-20 μg/mL. The sirtuin pathway activator may comprise nicotinamide riboside, nicotinamide mononucleotide, nicotinic acid, or a nicotinic acid metabolite.

The desired circulating level of the composition can be, can be about, can be more than, can be more than about, can be less than, or can be less than about 40, 60, 80, 100, 120, 150, 200, 300, 400, 800, 1600, 3000, or 5000 nM or more of the therapeutic agent. The selected dose can be chosen based on the characteristics of the subject, such as weight, height, ethnicity, or genetics.

In some embodiments, a composition comprises flavonoid or flavonoid derivative and sirtuin pathway activator in amounts that are effective to achieve a circulating level of or of about 0.3-1 mM flavonoid and of or of about 1-100 nM sirtuin pathway activator in a subject.

An oral dosing of or of about 1,125 mg flavonoid or flavonoid derivative can achieve a circulating level of flavonoid or flavonoid derivative in a subject that is or is about 0.5 mM flavonoid or flavonoid derivative. An oral dosing of about 300 mg flavonoid or flavonoid derivative can achieve a circulating level of flavonoid or flavonoid derivative in a subject that is or is about 0.25 mM.

An oral dosing of or of about 3,000 mg of sirtuin pathway activator can achieve a circulating level of sirtuin pathway activator in a subject that is or is about 10 μM sirtuin pathway activator. An oral dosing of or of about 50 mg of sirtuin pathway activator can achieve a circulating level of sirtuin pathway activator in a subject that is or is about 10-100 nM of sirtuin pathway activator. The sirtuin pathway activator may comprise nicotinamide riboside, nicotinamide mononucleotide, nicotinic acid, or a nicotinic acid metabolite.

An oral dosing of or of about 1100 mg of therapeutic agent (e.g., resveratrol) can achieve a circulating level of resveratrol in a subject that is or is about 0.5 mM of the therapeutic agent. An oral dosing of or of about 50 mg of resveratrol can achieve a circulating level of resveratrol in a subject that is or is about 200 nM resveratrol.

In some embodiments, the compositions can be formulated to achieve a desired circulating molar or mass ratios achieved after administration one or more compositions to a subject. The compositions can be a combination composition described herein. The molar ratio can be adjusted to account for the bioavailability, the uptake, and the metabolic processing of the one or more components of a combination composition. For example, if the bioavailability of a component is low, then the molar amount of a that component can be increased relative to other components in the combination composition. In some embodiments, the circulating molar or mass ratio is achieved within or within about 0.1, 0.5, 0.75, 1, 3, 5, or 10, 12, 24, or 48 hours after administration. The circulating molar or mass ratio can be maintained for a time period of or of about or of greater than, or of greater than about 0.1, 1, 2, 5, 10, 12, 18, 24, 36, 48, 72, or 96 hours.

In some embodiments, the circulating molar ratio of flavonoid or flavonoid derivative to sirtuin pathway activator is, is about, is less than, is less than about, is greater than, or is greater than about 1, 5, 10, 20, 50, 100, 500, 1000, 5000, or 10000. In some embodiments, the circulating molar ratio of sirtuin pathway activator to resveratrol is, is about, is less than, is less than about, is greater than, or is greater than about 0.01, 0.05, 0.1, 0.5, 1, 5, 10, 20, 50, or 100. The sirtuin pathway activator may comprise nicotinamide riboside, nicotinamide mononucleotide, nicotinic acid, or a nicotinic acid metabolite.

### Dosing Forms

The compositions described herein can be compounded into a variety of different dosage forms. It can be used orally as a tablet, a capsule, a pill, a granule, an emulsion, a gel, a plurality of beads encapsulated in a capsule, a powder, a suspension, a liquid, a semi-liquid, a semi-solid, a syrup, a slurry, a chewable form, caplets, soft gelatin capsules, lozenges or solution. Alternatively, the compositions can be formulated for inhalation or for intravenous delivery. The compositions can also be formulated as a nasal spray or for injection when in solution form. In some embodiments, the composition can be a liquid composition suitable for oral consumption.

Compositions formulated for inhalation can be packaged in an inhaler using techniques known in the art. An inhaler can be designed to dispense 0.25, 0.5, or 1 unit dose per inhalation. An inhaler can have a canister that holds the subject composition formulated for inhalation, a metering valve that allows for a metered quantity of the formulation to be dispensed with each actuation, and an actuator or mouthpiece that allows for the device to be operated and direct the subject composition into the subject's lungs. The formulated composition can include a liquefied gas propellant and possibly stabilizing excipients. The actuator can have a mating discharge nozzle that connects to the canister and a dust cap to prevent contamination of the actuator. Upon actuation, the subject composition can be volatized, which results in the formation of droplets of the subject composition. The droplets can rapidly evaporate resulting in micrometer-sized particles that are then inhaled by the subject. Inhalers and methods for formulating compositions for inhalation are described in are described in U.S. Pat. Nos. 5,069,204, 7,870,856 and U.S. Patent Application No. 2010/0324002, which are incorporated herein by reference in its entirety.

Compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, including liquid dosage forms (e.g., a suspension or slurry), and oral solid dosage forms (e.g., a tablet or bulk powder). Oral dosage forms can be formulated as tablets, pills, dragees, capsules, emulsions, lipophilic and hydrophilic suspensions, liquids, gels, syrups, slurries, suspensions and the like, for oral ingestion by an individual or a patient to be treated. Such dosage forms can be prepared by any of the methods of formulation. For example, the active ingredients can be brought into association with a carrier, which constitutes one or more necessary ingredients. Capsules suitable for oral administration include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. Optionally, the inventive composition for oral use can be obtained by mixing a composition a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The liquid forms, in which the formulations disclosed herein can be incorporated for administration orally or by injection, include aqueous solution, suitably flavored syrups, aqueous or oil suspensions, and flavored emulsions with edible oils such as cottonseed oil, sesame oil, coconut oil, or peanut oil as well as elixirs and similar pharmaceutical vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin.

A subject can be treated by combination of an injectable composition and an orally ingested composition.

Liquid preparations for oral administration can take the form of, for example, solutions, syrups or suspensions, or they can be presented as a dry product for reconstitution with water or other suitable vehicles before use. Such liquid preparations can be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

The preparation of pharmaceutical compositions of this invention, including oral and inhaled formulations, can be conducted in accordance with generally accepted procedures for the preparation of pharmaceutical preparations. See, for example, Remington's Pharmaceutical Sciences 18th Edition (1990), E. W. Martin ed., Mack Publishing Co., PA. Depending on the intended use and mode of administration, it can be desirable to process the magnesium-counter ion compound further in the preparation of pharmaceutical compositions. Appropriate processing can include mixing with appropriate non-toxic and non-interfering components, sterilizing, dividing into dose units, and enclosing in a delivery device.

This invention further encompasses anhydrous compositions and dosage forms comprising an active ingredient, since water can facilitate the degradation of some compounds. For example, water can be added (e.g., 5%) in the arts as a means of simulating long-term storage in order to determine characteristics such as shelf-life or the stability of formulations over time. Anhydrous compositions and dosage forms of the invention can be prepared using anhydrous or low moisture containing ingredients and low moisture or low humidity conditions. Compositions and dosage forms of the invention which contain lactose can be made anhydrous if substantial contact with moisture and/or humidity during manufacturing, packaging, and/or storage is expected. An anhydrous composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastic or the like, unit dose containers, blister packs, and strip packs.

An ingredient described herein can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Binders suitable for use in dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Lubricants which can be used to form compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the composition.

Lubricants can be also be used in conjunction with tissue barriers which include, but are not limited to, polysaccharides, polyglycans, seprafilm, interceed and hyaluronic acid.

Disintegrants can be used in the compositions of the invention to provide tablets that disintegrate when exposed to an aqueous environment. Too much of a disintegrant can produce tablets which can disintegrate in the bottle. Too little can be insufficient for disintegration to occur and can thus alter the rate and extent of release of the active ingredient(s) from the dosage form. Thus, a sufficient amount of disintegrant that is neither too little nor too much to detrimentally alter the release of the active ingredient(s) can be used to form the dosage forms of the compounds disclosed herein. The amount of disintegrant used can vary based upon the type of formulation and mode of administration, and can be readily discernible to those of ordinary skill in the art. About 0.5 to about 15 weight percent of disintegrant, or about 1 to about 5 weight percent of disintegrant, can be used in the pharmaceutical composition. Disintegrants that can be used to form compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Examples of suitable fillers for use in the compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

When aqueous suspensions and/or elixirs are desired for oral administration, the active ingredient therein can be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

In one embodiment, the composition can include a solubilizer to ensure good solubilization and/or dissolution of the compound of the present invention and to minimize precipitation of the compound of the present invention. This can be especially important for compositions for non-oral use, e.g., compositions for injection. A solubilizer can also be added to increase the solubility of the hydrophilic drug and/or other components, such as surfactants, or to maintain the composition as a stable or homogeneous solution or dispersion.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof. A non-exhaustive list of examples of excipients includes monoglycerides, magnesium stearate, modified food starch, gelatin, microcrystalline cellulose, glycerin, stearic acid, silica, yellow beeswax, lecithin, hydroxypropylcellulose, croscarmellose sodium, and crospovidone.

The compositions described herein can also be formulated as extended-release, sustained-release or time-release such that one or more components are released over time. Delayed release can be achieved by formulating the one or more components in a matrix of a variety of materials or by microencapsulation. The compositions can be formulated to release one or more components over a time period of 1, 4, 6, 8, 12, 16, 20, 24, 36, or 48 hours. The release of the one or more components can be at a constant or changing rate.

In some embodiments, a subject composition described herein can be formulated in as matrix pellets in which particles of the subject composition are embedded in a matrix of water-insoluble plastic and which are enclosed by a membrane of water-insoluble plastic containing embedded particles of lactose, produces and maintains plasma levels of the subject composition within the targeted therapeutic range. In other embodiments, a subject composition can be formulated as a sustained release tablet obtained by coating core granules composed mainly of the subject composition with a layer of a coating film composed of a hydrophobic material and a plastic excipient and optionally containing an enteric polymer material to form coated granules and then by compressing the coated granules together with a disintegrating excipient. Sustained release formulations are described in U.S. Pat. Nos. 4,803,080, and 6,426,091, which are herein incorporated by reference in its entirety.

Using the controlled release dosage forms provided herein, the one or more cofactors can be released in its dosage form at a slower rate than observed for an immediate release formulation of the same quantity of components. In some embodiments, the rate of change in the biological sample measured as the change in concentration over a defined time period from administration to maximum concentration for a controlled release formulation is less than or less than about 80%, 70%, 60%, 50%, 40%, 30%, 20%, or 10% of the rate of the immediate release formulation. Furthermore, in some embodiments, the rate of change in concentration over time is less than or less than about 80%, 70%, 60%, 50%, 40%, 30%, 20%, or 10% of the rate for the immediate release formulation.

In some embodiments, the rate of change of concentration over time is reduced by increasing the time to maximum concentration in a relatively proportional manner. For example, a two-fold increase in the time to maximum concentration can reduce the rate of change in concentration by approximately a factor of 2. As a result, the one or more cofactors can be provided so that it reaches its maximum concentration at a rate that is significantly reduced over an immediate release dosage form. The compositions of the present invention can be formulated to provide a shift in maximum concentration by 24 hours, 16 hours, 8 hours, 4 hours, 2 hours, or at least 1 hour. The associated reduction in rate of change in concentration can be by a factor of or of about 0.05, 0.10, 0.25, 0.5 or at least 0.8. In certain embodiments, this is accomplished by releasing less than or less than about 30%, 50%, 75%, 90%, or 95% of the one or more cofactors into the circulation within one hour of such administration.

Optionally, the controlled release formulations exhibit plasma concentration curves having initial (e.g., from 2 hours after administration to 4 hours after administration) slopes less than 75%, 50%, 40%, 30%, 20% or 10% of those for an immediate release formulation of the same dosage of the same cofactor.

In some embodiments, the rate of release of the cofactor as measured in dissolution studies is less than or less than about 80%, 70%, 60% 50%, 40%, 30%, 20%, or 10% of the rate for an immediate release formulation of the same cofactor over the first 1, 2, 4, 6, 8, 10, or 12 hours.

The controlled release formulations provided herein can adopt a variety of formats. In some embodiments, the formulation is in an oral dosage form, including liquid dosage forms (e.g., a suspension or slurry), and oral solid dosage forms (e.g., a tablet or bulk powder), such as, but not limited to those, those described herein.

The controlled release tablet of a formulation disclosed herein can be of a matrix, reservoir or osmotic system. Although any of the three systems is suitable, the latter two systems can have more optimal capacity for encapsulating a relatively large mass, such as for the inclusion of a large amount of a single cofactor, or for inclusion of a plurality of cofactors, depending on the genetic makeup of the individual. In some embodiments, the slow-release tablet is based on a reservoir system, wherein the core containing the one or more cofactors is encapsulated by a porous membrane coating which, upon hydration, permits the one or more cofactors to diffuse through. Because the combined mass of the effective ingredients is generally in gram quantity, an efficient delivery system can provide optimal results.

Thus, tablets or pills can also be coated or otherwise compounded to provide a dosage form affording the advantage of prolonged action. For example, the tablet or pill can comprise an inner dosage an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings such materials including a number of polymeric acids and mixtures of polymeric acids with such materials as shellac, cetyl alcohol and cellulose acetate. In some embodiments, a formulation comprising a plurality of cofactors can have different cofactors released at different rates or at different times. For example, there can be additional layers of cofactors interspersed with enteric layers.

Methods of making sustained release tablets are known in the art, e.g., see U.S. Patent Publications 2006/051416 and 2007/0065512, or other references disclosed herein. Methods such as described in U.S. Pat. Nos. 4,606,909, 4,769,027, 4,897,268, and 5,395,626 can be used to prepare sustained release formulations of the one or more cofactors determined by the genetic makeup of an individual. In some embodiments, the formulation is prepared using OROS® technology, such as described in U.S. Pat. Nos. 6,919,373, 6,923,800, 6,929,803, and 6,939,556. Other methods, such as described in U.S. Pat. Nos. 6,797,283, 6,764,697, and 6,635,268, can also be used to prepare the formulations disclosed herein.

In some embodiments, the compositions can be formulated in a food composition. For example, the compositions can be a beverage or other liquids, solid food, semi-solid food, with or without a food carrier. For example, the compositions can include a black tea supplemented with any of the compositions described herein. The composition can be a dairy product supplemented any of the compositions described herein. In some embodiments, the compositions can be formulated in a food composition. For example, the compositions can comprise a beverage, solid food, semi-solid food, or a food carrier.

In some embodiments, liquid food carriers, such as in the form of beverages, such as supplemented juices, coffees, teas, sodas, flavored waters, and the like can be used. For example, the beverage can comprise the formulation as well as a liquid component, such as various deodorant or natural carbohydrates present in conventional beverages. Examples of natural carbohydrates include, but are not limited to, monosaccharides such as, glucose and fructose; disaccharides such as maltose and sucrose; conventional sugars, such as dextrin and cyclodextrin; and sugar alcohols, such as xylitol and erythritol. Natural deodorant such as taumatin, stevia extract, levaudioside A, glycyrrhizin, and synthetic deodorant such as saccharin and aspartame can also be used. Agents such as flavoring agents, coloring agents, and others can also be used. For example, pectic acid and the salt thereof, alginic acid and the salt thereof, organic acid, protective colloidal adhesive, pH controlling agent, stabilizer, a preservative, glycerin, alcohol, or carbonizing agents can also be used. Fruit and vegetables can also be used in preparing foods or beverages comprising the formulations discussed herein.

Alternatively, the compositions can be a snack bar supplemented with any of the compositions described herein. For example, the snack bar can be a chocolate bar, a granola bar, or a trail mix bar. In yet another embodiment, the present dietary supplement or food compositions are formulated to have suitable and desirable taste, texture, and viscosity for consumption. Any suitable food carrier can be used in the present food compositions. Food carriers of the present invention include practically any food product. Examples of such food carriers include, but are not limited to food bars (granola bars, protein bars, candy bars, etc.), cereal products (oatmeal, breakfast cereals, granola, etc.), bakery products (bread, donuts, crackers, bagels, pastries, cakes, etc.), beverages (milk-based beverage, sports drinks, fruit juices, alcoholic beverages, bottled waters), pastas, grains (rice, corn, oats, rye, wheat, flour, etc.), egg products, snacks (candy, chips, gum, chocolate, etc.), meats, fruits, and vegetables. In an embodiment, food carriers employed herein can mask the undesirable taste (e.g., bitterness). Where desired, the food composition presented herein exhibit more desirable textures and aromas than that of any of the components described herein. For example, liquid food carriers can be used according to the invention to obtain the present food compositions in the form of beverages, such as supplemented juices, coffees, teas, and the like. In other embodiments, solid food carriers can be used according to the invention to obtain the present food compositions in the form of meal replacements, such as supplemented snack bars, pasta, breads, and the like. In yet other embodiments, semi-solid food carriers can be used according to the invention to obtain the present food compositions in the form of gums, chewy candies or snacks, and the like.

The dosing of the combination compositions can be administered at, at about, at less than, at less than about, at more than, or at more than about 1, 2, 3, 4, 5, 6, 7, 8, 9, 10 or more times daily. A subject can receive dosing for a period of exactly, about, less than, less than about, greater than, or greater than about 1, 2, 3, 4, 5, 6, 7, 8, 9, 10, 11, 12, 13, 14 or more days, weeks or months. A unit dose can be a fraction of the daily dose, such as the daily dose divided by the number of unit doses to be administered per day. A unit dose can be a fraction of the daily dose that is the daily dose divided by the number of unit doses to be administered per day and further divided by the number of unit doses (e.g. tablets) per administration. The number of unit doses per administration can be exactly, about, less than, less than about, more than, or more than about 1, 2, 3, 4, 5, 6, 7, 8, 9, 10, or more. The number of doses per day can be exactly, about, less than, less than about, more than, or more than about 1, 2, 3, 4, 5, 6, 7, 8, 9, 10, or more. The number of unit doses per day can be determined by dividing the daily dose by the unit dose, and can be exactly, about, less than, less than about, more than, or more than about 1, 2, 3, 4, 5, 6, 7, 8, 9, 10, 11, 12, 13, 14, 15, 6, 17, 18, 19, 20, or more unit doses per day. For example, a unit dose can be or can be about ½, ⅓, ¼, ⅕, ⅙, 1/7, ⅛, 1/9, 1/10. A unit dose can be or can be about one-third of the daily amount and administered to the subject three times daily. A unit dose can be or can be about one-half of the daily amount and administered to the subject twice daily. A unit dose can be or can be about one-fourth of the daily amount with two unit doses administered to the subject twice daily. In some embodiments, a unit dose comprises exactly, about, less than, less than about, more than, or more than about 50 mg resveratrol. In some embodiments, a unit dose comprises exactly, about, less than, less than about, more than, or more than about 550 mg naringenin. In some embodiments, a unit dose comprises exactly, about, less than, less than about, more than, or more than about 200 mg of one or more flavonoid derivatives.

In some embodiments, a unit dose (e.g. a unit dose comprising one or more flavonoid derivatives, such as 8-prenylnaringenin) is administered as one unit dose two times per day. A unit dose can comprise more than one capsule, tablet, vial, or entity.

Compositions disclosed herein can further comprise a flavorant and can be a solid, liquid, gel or emulsion.

When the subject composition administered further comprises one or more therapeutic agents, and the therapeutic agents have a shorter half-life than the flavonoid or flavonoid derivative, or the sirtuin pathway activator, the unit dose forms of the therapeutic agent and the flavonoid or flavonoid derivatives, or sirtuin pathway activator can be adjusted accordingly.

### Methods

The subject application provides methods of increasing sirtuin pathway output (including AMPK, a signaling molecule in the sirtuin pathway) in a subject. As described herein, the output of the sirtuin pathway can be characterized at the molecular level or by a resulting physiological effect. In some embodiments, the invention provides for methods of increasing fatty acid oxidation in a subject comprising the administration of a composition as disclosed herein to the subject. In various embodiments of the invention, a composition is administered to the subject in an amount that delivers synergizing amounts of one or more flavonoids or flavonoid derivatives and one or more sirtuin pathway activator sufficient to increase fatty acid oxidation, fat loss or weight loss, thermogenesis, insulin sensitivity, and/or adipocyte beiging within the cells of the subject.

The methods described herein can be useful for a variety of applications. These applications include (a) an increase in sirtuin-pathway output, (b) an increase in mitochondrial biogenesis, (c) an increase in the formation of new mitochondria, (d) an increase in mitochondrial functions, (e) an increase in fatty acid oxidation, (f) an increase in heat generation, (g) an increase in insulin sensitivity, (h) an increase in glucose uptake, (i) a decrease in weight, and (j) a decrease in adipose weight or volume. Any of these applications can be achieved by administering one or more compositions described herein.

Accordingly, the invention provides a method for administering a composition comprising (a) one or more types of flavonoids or flavonoid derivatives and (b) a sirtuin-pathway activator present in a sub-therapeutic amount, wherein the composition is synergistically effective in increasing the sirtuin-pathway output by at least about 5 fold as compared to that of component (b) when it is being used alone.

The output of the pathways can be measured using one or more methods, disclosed herein and/or known in the art. For example, fatty acid oxidation can be determined by measuring oxygen consumption, or ³H-labeled palmitate oxidation. Mitochondrial biogenesis can be measured using a mitochondrial probe by using fluorescence. AMPK activity can be determined by measuring AMPK phosphorylation via an ELISA assay or by Western blot. Sirt1 activity can be determined by measuring deacetylation of a substrate, which can be detected using a fluorophore.

An increase in sirt1, sirt2, or sirt3 is observed by applying a corresponding substrate in a deacylation assay conducted in vitro. The substrate for measuring SIRT1 activity can be any substrate known in the art (for example a peptide containing amino acids 379-382 of human p53 (Arg-His-Lys-Lys[Ac]). The substrate for measuring SIRT3 activity can be any substrate known in the art (for example a peptide containing amino acids 317-320 of human p53 (Gln-Pro—Lys-Lys[Ac])). In some instances, the increase in sirt activity in one or more assays conducted in the presence of one or more combination compositions described herein results in an activity increase of at least or at least about 1, 2, 3, 5, or 10 fold, as compared to the activity measured in the presence of only one component of the combination compositions. For example, the use of a combination composition comprising (a) a sirtuin pathway activator (such as nicotinamide riboside) and (b) a flavonoid or flavonoid derivative (such as naringenin) results in an increase in sirt3 activity by at least about 5 fold as compared to the activity measured in the presence of (a) or (b) alone. Also, the use of a combination composition comprising naringenin and nicotinamide riboside results in an increase in sirt1 activity that is 1.5, 2, 5 or 10 fold greater than the activity measured in the presence of only naringenin or nicotinamide riboside.

The invention provides a method for administering a composition comprising: (a) one or more types of flavonoids or flavonoid derivatives, and (b) a sirtuin-pathway activator, wherein molar ratio of component (a) to (b) in said composition is greater than or greater than about 20, and wherein the composition when administered to a subject in need thereof synergistically enhances a physiological effect selected from mitochondrial biogenesis, fatty acid oxidation, thermogenesis, adipocyte beiging, weight loss or fat loss, or insulin sensitivity as measured by a decrease in weight gain of a subject, a decrease in visceral adipose volume of a subject, an increase in fat oxidation of a subject, an increase in insulin sensitivity of a subject, an increase of glucose uptake in muscle of a subject, and/or an increase in body temperature.

The invention provides a method for administering a food composition comprising: (a) one or more types of flavonoids or flavonoid derivatives; (b) a sirtuin pathway activator, wherein (a) and (b) are present in an amount that synergistically effect a decrease in weight gain of a subject, a decrease in visceral adipose volume of a subject, an increase in fat oxidation of a subject, an increase in insulin sensitivity of a subject, an increase of glucose uptake in muscle of a subject, and/or an increase in body temperature; and (c) a food carrier.

The invention provides a method for administering a composition comprising: a synergistically effective amount of (a) one or more types of flavonoids or flavonoid derivatives; and (b) a sirtuin-pathway activator, wherein the composition is substantially free of non-branched amino acids, wherein the combination when administered to a subject in need thereof enhances a physiological effect selected from mitochondrial biogenesis, fatty acid oxidation, thermogenesis, adipocyte beiging, weight loss or fat loss, or insulin sensitivity to a greater degree as compared to administering to a subject component (a) or component (b) alone, and wherein the physiological effect is measured by a decrease in weight of a subject, a decrease in visceral adipose volume of a subject, an increase in fat oxidation of a subject, an increase in insulin sensitivity of a subject, an increase in thermogenesis of a subject, and increase in adipocyte beiging of a subject, an increase of glucose uptake in muscle of a subject, and/or an increase in body temperature.

The invention provides a method for administering a composition comprising: (a) one or more types of flavonoids or flavonoid derivatives, and (b) a signaling molecule downstream of PGC1α in a sirtuin-signaling pathway.

The invention provides for a method of enhancing fat oxidation, thermogenesis, insulin sensitivity, and/or adipocyte beiging in a subject in need thereof comprising administering to the subject any of the compositions described herein over a time period, wherein the fat oxidation, thermogenesis, insulin sensitivity, and/or adipocyte beiging in the subject is increased over the time period. The fat oxidation, thermogenesis, insulin sensitivity, and/or adipocyte beiging can be increased by about or greater than or greater than about 5, 10, 15, 20, 50, 100, 200, or 500%.

The invention provides for a method of reducing an inflammatory response in a subject in need thereof comprising administering to the subject a composition any of the compositions described herein over a time period, wherein the inflammatory response in the subject is reduced over the time period. The inflammatory response can be decreased by or by about or greater than, or greater than about 5, 10, 15, 20, 50, or 100%.

Inflammatory marker and cytokine levels, including but not limited to IL-6, adiponectin, TNF-α and CRP levels in plasma can determined by immune assays, such as ELISA (Assay Designs, Ann Arbor, Mich.; Linco Research, St. Charles, Mo.; and Bioscience, San Diego, Calif.).

The invention provides for a method of increasing or maintaining body temperature in a subject comprising administering to the subject a composition any of the compositions described herein over a time period, wherein the body temperature in the subject is increased over the time period. The body temperature can be increased by or by about or by greater than or greater than about 1, 2, 3, 4, 5, 10, 15, or 20%.

The invention provides for a method of treating diabetes, comprising administering to the subject any of the compositions described herein over a time period, wherein the insulin sensitivity in the subject is increased over the time period. Insulin sensitivity can be increased by or by about or by greater than or by greater than about 1, 2, 3, 5, 10, 20, 50, 100, or 200%. In some embodiments, a flavonoid (or a flavonoid derivative) and/or a sirtuin pathway activator are administered in an amount that reduces the therapeutically effective dose of metformin for a subject. In some embodiments, the therapeutically effective dose of metformin is reduced by or by about or by more than or by more than about 50%, 60%, 70%, 80%, 90%, 95%, 97.5%, 99.9%, 99.99%, or more. In some embodiments, administration of compositions of the invention reduces body fat (e.g. visceral fat) by or by about or by more than or by more than about 5%, 10%, 15%, 20%, 25%, 50%, or more.

Insulin sensitivity can be measured using a variety of techniques, including HOMA_{IR}. HOMA_{IR}, which is the homeostasis model assessment of insulin resistance can be used as a screening index of changes in insulin sensitivity. HOMA_{IR} can be calculated via standard formula from fasting plasma insulin and glucose as follows: HOMA_{IR}=[Insulin (uU/mL)×glucose (mM)]/22.5. Insulin sensitivity can also be measured by measurements of fasting insulin, measuring changes in 60-minute glucose or insulin via a glucose tolerance test (GTT), or changes in the glucose or insulin area under the curve in a GTT.

In some embodiments, insulin signaling can also be measured. Insulin signaling can be measured by measuring total and phosphorylated Akt, GSK-3β, IGF-1R, IR, IRS-1, p70S6K and PRAS40 in tissue lysates via the Luminex Kits “Akt Pathway Total 7-Plex Panel” (Cat # LHO0002) and “Akt Pathway Phospho 7-Plex Panel” (Cat # LH00001) from Invitrogen Life Science. The subject application also provides methods of increasing mitochondrial biogenesis in a subject comprising the administration of a composition disclosed herein to a subject.

In various embodiments of the invention, a composition is administered to the subject in an amount that delivers synergizing amounts of flavonoid or flavonoid derivative and sirtuin pathway activator sufficient to increase mitochondrial biogenesis within the cells of the subject. Another embodiment provides for the administration of a composition comprising synergizing amounts of naringenin and nicotinamide riboside to the subject in an amount sufficient to increase mitochondrial biogenesis within the cells of the subject. Yet other embodiments provide for the administration of a composition comprising synergizing amounts of naringenin, nicotinamide riboside, and resveratrol to a subject in an amount sufficient to increase mitochondrial biogenesis in the subject. Mitochondrial biogenesis and fat oxidation may be induced in various cells, including muscle cells and adipocytes.

Another aspect of the invention provides methods of reducing weight gain or reducing adipose volume in a subject comprising the administration of compositions disclosed herein. Body weight can be measured with a calibrated scale and height measured with a wall-mounted stadiometer, and body mass index can be calculated via standard equation (kg/m²). Fat mass can be assessed via dual-energy X-ray absorptiometry at baseline, and 12 and 24 weeks. A LUNAR Prodigy dual-energy X-ray absorptiometry system (GE Healthcare, Madison, Wis.), or any other X-ray absorptiometry system known in the art, can be maintained and calibrated for use. A spine phantom can be assessed every day to determine whether any drift in the machine occurred, followed by a daily calibration block.

In this aspect of the invention, a composition is administered to the subject in an amount that delivers synergizing amounts of flavonoid or flavonoid derivative and sirtuin pathway activator sufficient to reduce weight gain in a subject. Another embodiment provides for the administration of a composition comprising synergizing amounts of naringenin and nicotinamide riboside to the subject in an amount sufficient to reduce weight gain in the subject. Yet other embodiments provide for the administration of a composition comprising synergizing amounts of naringenin, nicotinamide riboside, and resveratrol to a subject in an amount sufficient to reduce weight gain in the subject.

Administration of compositions disclosed herein that increase SIRT1 and SIRT3 activity may be useful in any subject in need of metabolic activation of adipocytes or one or more of their muscles, e.g., skeletal muscle, smooth muscle or cardiac muscle or muscle cells thereof. A subject may be a subject having cachexia or muscle wasting. Increasing SIRT3 activity may also be used to increase or maintain body temperature, e.g., in hypothermic subjects and increasing SIRT1 activity is beneficial for treating diabetes (type 2 diabetes) and impaired glucose tolerance and reducing inflammatory responses in a subject.

Increasing SIRT3 activity may also be used for treating or preventing cardiovascular diseases, reducing blood pressure by vasodilation, increasing cardiovascular health, and increasing the contractile function of vascular tissues, e.g., blood vessels and arteries (e.g., by affecting smooth muscles). Generally, activation of SIRT3 may be used to stimulate the metabolism of adipocytes or any type of muscle, e.g., muscles of the gut or digestive system, or the urinary tract, and thereby may be used to control gut motility, e.g., constipation, and incontinence. SIRT3 activation may also be useful in erectile dysfunction. It may also be used to stimulate sperm motility, e.g., and be used as a fertility drug. Other embodiments in which it would be useful to increase SIRT3 include repair of muscle, such as after a surgery or an accident, increase of muscle mass; and increase of athletic performance.

Thus, the invention provides methods in which beneficial effects are produced by contacting one or more muscle cells with an agent that increases the protein or activity level of SIRT3 in the cell. These methods effectively facilitate, increase or stimulate one or more of the following: mimic the benefits of calorie restriction or exercise in the muscle cell, increase mitochondrial biogenesis or metabolism, increase mitochondrial activity and/or endurance in the muscle cell, sensitize the muscle cell to glucose uptake, increase fatty acid oxidation in the muscle cell, increase PGC-1α and/or UCP3 and/or GLUT4 expression in the muscle cell, and activate AMP activated protein kinase (AMPK) in the muscle cell. Various types of muscle cells can be contacted in accordance with the invention. In some embodiments, the muscle cell is a skeletal muscle cell. In certain embodiments, the muscle cell is a cell of a slow-twitch muscle, such as a soleus muscle cell.

Resting metabolic rate (RMR)/Substrate Oxidation is measured by indirect calorimetry using the open circuit technique between the hours of 6 AM and 10 AM after a 12-hour fast and 48-hour abstention from exercise utilizing a SensorMedics Vmax 29n metabolic cart (Sensor Medics, Anaheim, Calif.). Following a urinary void, the participant rests quietly for 30 minutes in an isolated room with temperature controlled (21-24° C.) environment. The subject is then placed in a ventilated hood for a minimum of 30 minutes, until steady state is achieved. Criteria for a valid measurement can be a minimum of 15 minutes of steady state, with steady state determined as less than 10% fluctuation in minute ventilation and oxygen consumption and less than 5% fluctuation in respiratory quotient. Metabolic rate is calculated using the Weir equation, respiratory quotient (RQ) is calculated as CO₂ production/O₂ consumption, and substrate oxidation is calculated from RQ after correction for urinary nitrogen losses. Whole body fatty acid oxidation may be measured by respiratory calorimetry measurements. Changes in oxygen consumption and/or changes in RQ may be used to estimate changes in the rate of fatty acid oxidation.

Glucose uptake can be measured using in vivo or in vitro techniques. For example, glucose uptake can be measured in vivo using a PET scan in conjunction with labeled glucose or glucose analog. Measurements of glucose uptake can be quantified from the PET scan or by any other technique known in the art. In some embodiments, the glucose uptake can be measured by quantitation of exogenously administered 18-F-deoxyglucose uptake via PET.

ROS/Oxidative Stress can be measured by drawing blood into EDTA-treated tubes, centrifuging to separate plasma, and aliquoting samples for individual assays. Plasma can be maintained at −80° C. under nitrogen to prevent oxidative changes prior to measurements. Plasma malonaldehyde (MDA) can be measured using a fluorometric assay, and plasma 8-isoprostane F_{2α} can be measured by ELISA (Assay Designs, Ann Arbor, Mich.).

Another embodiment provides for the administration of a composition comprising synergizing amounts of a flavonoid or flavonoid derivatives and nicotinic acid and/or nicotinamide riboside and/or nicotinamide mononucleotide and/or nicotinic acid metabolites. Another embodiment provides for the administration of a composition comprising synergizing amounts of naringenin and nicotinamide riboside to the subject in an amount sufficient to increase fatty acid oxidation within the cells of the subject. Yet other embodiments provide for the administration of a composition comprising synergizing amounts of naringenin, nicotinamide riboside, and resveratrol to a subject in an amount sufficient to increase fatty acid oxidation in the subject.

The compositions can be administered to a subject orally or by any other methods. Methods of oral administration include administering the composition as a liquid, a solid, or a semi-solid that can be taken in the form of a dietary supplement or a food stuff.

The compositions can be administered periodically. For example, the compositions can be administered one, two, three, four times a day, or even more frequent. The subject can be administered every 1, 2, 3, 4, 5, 6 or 7 days. In some embodiments, the compositions are administered three times daily. The administration can be concurrent with meal time of a subject. The period of treatment or diet supplementation can be for or for about 1, 2, 3, 4, 5, 6, 7, 8, or 9 days, 2 weeks, 1-11 months, or 1 year, 2 years, 5 years or even longer. In some embodiments of the invention, the dosages that are administered to a subject can change or remain constant over the period of treatment. For example, the daily dosing amounts can increase or decrease over the period of administration.

The length of the period of administration and/or the dosing amounts can be determined by a physician, a nutritionist, or any other type of clinician. The physician, nutritionist, or clinician can observe the subject's response to the administered compositions and adjust the dosing based on the subject's performance. For example, dosing for subjects that show reduced effects in energy regulation can be increased to achieve desired results.

In some embodiments, the compositions administered to a subject can be optimized for a given subject. For example, the ratio of flavonoid or flavonoid derivative to a sirtuin pathway activator or the particular components in a combination composition can be adjusted. The ratio and/or particular components can be selected after evaluation of the subject after being administered one or more compositions with varying ratios of flavonoid or flavonoid derivative to a sirtuin pathway activator or varying combination composition components.

Another aspect of the invention provides for achieving desired effects in one or more subjects after administration of a combination composition described herein for a specified time period.

After a period of 6 weeks of administration of the composition, a combination composition comprising (a) a dosing level of flavonoid or flavonoid derivative and a dosing level of a sirtuin pathway activator or (b) a dosing level of naringenin and a dosing level of nicotinamide riboside can reduce weight gain in the one or more subjects by at least or at least about 10, 15, 20, or 20.5%. The p-value can be less than 0.05 (e.g. less than about 0.05, 0.03, 0.02, 0.01, 0.001, 0.0001, or lower). The one or more subjects treated with the same dosing level of one of the components (e.g., naringenin or nicotinamide riboside) may have insignificant weight reduction, or a weight reduction that is less than or less than about 0, 5, or 10%.

After a period of 2 weeks of administration, a composition comprising a dosing level of flavonoids or flavonoid derivatives and sirtuin pathway activator can increase whole body fat oxidation in the one or more subjects by at least or at least about 10, 15, or 20%. The p-value can be less than 0.05 (e.g. less than about 0.05, 0.03, 0.02, 0.01, 0.001, 0.0001, or lower). The increase in whole body fat oxidation can be sustained while the subjects are administered the composition, or for a period of at least 2, 4, 6, 10, 13, 26, or 52 weeks. The one or more subjects treated with the same dosing level of one of the components (e.g., naringenin or nicotinamide riboside) may have insignificant increase in whole body fat oxidation, or an increase in whole body fat oxidation that is less than or less than about 0, 5, or 10%.

After a period of 2 weeks of administration, a composition comprising a dosing level of flavonoids or flavonoid derivatives and sirtuin pathway activator can increase the thermic effect of food in the one or more subjects by at least or at least about 10, 15, 17, or 20%. The p-value can be less than 0.05 (e.g. less than about 0.05, 0.03, 0.02, 0.01, 0.001, 0.0001, or lower). The increase in the thermic effect of food can be sustained while the subjects are administered the composition, or for a period of at least 2, 4, 6, 10, 13, 26, or 52 weeks. The one or more subjects treated with the same dosing level of one of the components (e.g., naringenin or nicotinamide riboside) may have insignificant increase the thermic effect of food, or an increase the thermic effect of food that is less than or less than about 0, 5, or 10%.

After a period of 2 weeks of administration, a composition comprising a dosing level of flavonoids or flavonoid derivatives and sirtuin pathway activator can increase total energy expenditure in the one or more subjects by at least or at least about 10, 15, 17, or 20%. The p-value can be less than 0.05 (e.g. less than about 0.05, 0.03, 0.02, 0.01, 0.001, 0.0001, or lower). The increase total energy expenditure can be sustained while the subjects are administered the composition, or for a period of at least 2, 4, 6, 10, 13, 26, or 52 weeks. The one or more subjects treated with the same dosing level of one of the components (e.g., naringenin or nicotinamide riboside) may have insignificant increase total energy expenditure, or an increase total energy expenditure that is less than about 0, 5, or 10%.

The administration of a composition described herein, such as a combination composition, to a subject can allow for the regulation or maintenance of the subject's energy metabolism. The regulation or maintenance of energy metabolism can allow for a subject to experience a number of beneficial effects. These beneficial effects include a reduction in weight, a reduction in adipose tissue, an increase in fatty acid oxidation, a decrease in weight or adipose tissue weight, an increase in browning of adipose tissue (as indicated by one or more indicia of fat cell browning), an increase in insulin sensitivity, an increase in thermogenesis, a decrease in oxidative stress, and/or a decrease in inflammation. Compared to a baseline prior to treatment, these effects can result in an improvement of about or greater than or greater than about 5%, 10%, 15%, 20%, 30%, 40%, 50%, 75%, or more. In some embodiments, compared to a baseline prior to treatment, these effects can result in an improvement of, of about, greater than, or greater than about 100%, 125%, 150%, 200%, 250%, 300%, 400%, 500%, or more. Alternatively, administration of a composition described herein can allow for maintenance of the subject's weight, amount of adipose tissue, amount of fatty acid oxidation, level of insulin sensitivity, oxidative stress level, and/or level of inflammation. These amounts and/or levels can be maintained within about 0%, 1%, 5%, or 10% of the amounts and/or levels at the initiation of administration.

The invention provides for a method of treating subjects, comprising identifying a pool of subjects amenable to treatment. The identifying step can include one or more screening tests or assays. For example, subjects that are identified as diabetic or that have above average or significantly greater than average body mass indices and/or weight can be selected for treatment. The identifying step can include a genetic test that identifies one or more genetic variants that suggest that the subject is amenable to treatment. The identified subjects can then be treated with one or more compositions described herein. For example, they may be treated with a combination composition comprising a sirtuin pathway activator and a branched-chain amino acid.

In some embodiments, the compositions can be combined or mixed with a pharmaceutically active agent, a carrier, and/or an excipient. Examples of such components are described herein. The combined compositions can be formed into a unit dosage as tablets, capsules, gel capsules, slow-release tablets, or the like.

In some embodiments, the composition is prepared such that a solid composition containing a substantially homogeneous mixture of the one or more components is achieved, such that the one or more components are dispersed evenly throughout the composition so that the composition may be readily subdivided into equally effective unit dosage forms such as tablets, pills and capsules.

### Kits

The invention also provides kits. The kits include one or more compositions described herein, in suitable packaging, and can further comprise written material that can include instructions for use, discussion of clinical studies, listing of side effects, and the like. Such kits can also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information can be based on the results of various studies, for example, studies using experimental animals involving in vivo models and studies based on human clinical trials. A kit can comprise one or more unit doses described herein. In some embodiments, a kit comprises exactly, about, less than, less than about, more than, or more than about 1, 2, 3, 4, 5, 6, 7, 8, 9, 10, 11, 12, 13, 14, 15, 16, 17, 18, 19, 20, 30, 31, 60, 90, 120, 150, 180, 210, or more unit doses. Instructions for use can comprise dosing instructions, such as instructions to take 1, 2, 3, 4, 5, 6, 7, 8, 9, 10, or more unit doses 1, 2, 3, 4, 5, 6, 7, 8, 9, 10, or more times per day. For example, a kit can comprise a unit dose supplied as a tablet, with each tablet package separately, multiples of tablets packaged separately according to the number of unit doses per administration (e.g. pairs of tablets), or all tablets packaged together (e.g. in a bottle). As a further example, a kit can comprise a unit dose supplied as a bottled drink, the kit comprising 1, 2, 3, 4, 5, 6, 7, 8, 9, 10, 11, 12, 13, 14, 24, 28, 36, 48, 72, or more bottles.

The kit can further contain another agent. In some embodiments, the compound of the present invention and the agent are provided or packaged as separate compositions in separate containers within the kit. In some embodiments, the compound of the present invention and the agent are provided or packaged as a single composition within a container in the kit. Suitable packaging and additional articles for use (e.g., measuring cup for liquid preparations, foil wrapping to minimize exposure to air, and the like) are known in the art and can be included in the kit. Kits described herein can be provided, marketed and/or promoted to health providers, including physicians, nurses, pharmacists, formulary officials, and the like. Kits can also, in some embodiments, be marketed directly to the consumer.

In some embodiments, a kit can comprise a multi-day supply of unit dosages. The unit dosages can be any unit dosage described herein. The kit can comprise instructions directing the administration of the multi-day supply of unit dosages over a period of multiple days. The multi-day supply can be a one-month supply, a 30-day supply, or a multi-week supply. The multi-day supply can be a 90-day, 180-day, 3-month or 6-month supply. The kit can include packaged daily unit dosages, such as packages of 1, 2, 3, 4, or 5 unit dosages. The kit can be packaged with other dietary supplements, vitamins, and meal replacement bars, mixes, and beverages.

### EXAMPLES

### Example 1: Method for Cell Culture of Human Adipose Derived Stem Cells

Human adipose derived stem cells (hASC, LaCell, L.L.C., New Orleans, La.) from two different female overweight (BMI=26) donors were plated at a density of 10000 cells/cm² on 96-well plates and 24-well plates in preadipocyte medium (HyClone DMEM/F12 1:1, 10% fetal bovine serum (FBS), 1% Antibiotic/Antimycotic (Penicillin/Streptomycin/amphotericin)) at 37° C. in 5% CO₂. One day after reaching confluence, preadipocytes were induced to differentiate with a differentiation medium consisting of 70% Gibco DMEM (high glucose 4.5 g/L), 30% HyClone DMEM/F12 1:1 (3 g/L glucose), 3% FBS, 1% Antibiotic/Antimycotic, 1 μM Dexamethasone, 33 μM Biotin, 100 nM Insulin, 20 μM Pantothenate, 5 μM Rosiglitazone, 500 μM IBMX. Preadipocytes were maintained in this differentiation medium for 4 to 5 days and subsequently cultured in maintenance medium (70% Gibco DMEM high glucose, 30% HyClone DMEM/F12 1:1, 3% FBS, 1% Antibiotic/Antimycotic, 1 μM Dexamethasone, 33 μM Biotin, 100 nM Insulin, 20 μM Pantothenate) (=day 0 for experiments). After three days in maintenance medium, experiment medium (70% Gibco DMEM high glucose, 30% HyClone DMEM/F12 1:1, 3% Heat Inactivated FBS, 1% Antibiotic/Antimycotic, 33 μM Biotin, 100 nM Insulin, 20 μM Pantothenate) containing the treatments (8 μM Naringenin (Nar), 30 μM Nicotinamide Riboside (NR) or the combination) was administered for 6 days with fresh media change every two days.

### Example 2: Method for Western Blot of AMPK and SIRT1 Proteins

The Phospho-AMPKα (Thr172)-, AMPK- and Sirt1—antibody was obtained from Cell Signaling (Danvers, Mass.). hASC cells were grown on 24-well plates and treated as indicated. Proteins were extracted by using standard protocols and measured by BCA kit (Thermo Scientific). For Western blot, 20 μg of protein from the cell lysate was resolved on 10% Tris/HCL polyacrylamide gels (Criterion precast gel, Bio-Rad Laboratories, Hercules, Calif.), transferred to nitrocellulose membranes, incubated in blocking buffer (5% non-fat dry milk in 0.1% T-TBS) and then incubated with primary antibody (1:1000 dilution), washed and incubated with secondary horseradish peroxidase-conjugated antibody (anti-rabbit, 1:10.000 dilution). Visualization and chemiluminescent detection was conducted using BioRad ChemiDoc instrumentation and software (Bio-Rad Laboratories, Hercules, Calif.) and band intensity was assessed using Image Lab 4.0 (Bio-Rad Laboratories, Hercules, Calif.), with correction for background. Band intensities were normalized to stain-free blots to control for loading.

### Example 3: Method for Gene Expression

hASC cells were grown in a 96-well plate and treated as indicated. Cell Lysis and reverse transcription were performed using the TaqMan® Gene Expression Cells-to Ct™ Kit (Life Technologies, Cat #4399002) according to manufacturer's instructions. Gene expression was assessed by quantitative real-time polymerase chain reaction (qRT-PCR) using StepOnePlus™ PCR system (Thermo Fisher Scientific) and TaqMan Gene expression assays for UCP1 (Invitrogen, assay ID # Hs01084772_m1), Glut 4 (Invitrogen, assay ID # Hs00168966_m1), CPT1b (Invitrogen, assay ID # Hs0046298_s1), PRDM16 (Invitrogen, assay ID # Hs00223161_m1), AdipoQ (Invitrogen, assay ID # HS00605917_m1), ChREBP (Invitrogen, assay ID # HS00975714_m1), ATGL (Invitrogen, assay ID # Hs00982042), and RPL13A (Invitrogen, assay ID #Hs04194366_g1) as endogenous control.

### Example 4: Method of Fatty Acid Oxidation Measurement

The palmitate-stimulated oxygen consumption rate was measured with the XF 24 analyzer (Seahorse Bioscience, Billerica, Mass., USA) Cells were seeded and differentiated as described above. Media was then changed and the cells treated as indicated above, washed twice with non-buffered carbonate-free pH 7.4 low glucose (2.5 mM) DMEM containing carnitine (0.5 mM), equilibrated with 550 μL of the same media in a non-CO₂ incubator for 45 minutes, and then inserted into the instrument for 15 minutes of further equilibration, followed by O₂ consumption measurement. Three successive baseline measures at five-minute intervals were taken prior to injection of palmitate (200 μM final concentration). Four successive 5-minute measurements of O₂ consumption were then conducted, followed by 10 minute re-equilibration and another 3-4 5-minute measurements. This measurement pattern was then repeated over a 4-6 hour period. Data for each sample were normalized to the pre-palmitate injection baseline for that sample and expressed as % change from that baseline. Data were analyzed via one-way analysis of variance and least significant difference test was used to separate significantly different group means using GraphPad Prism version 7 (GraphPad Software, La Jolla Calif. USA, www.graphpad.com). All data are expressed as mean±SEM.

### Example 5: Gene Expression Results for UCP1 with Naringenin-NR Treatment

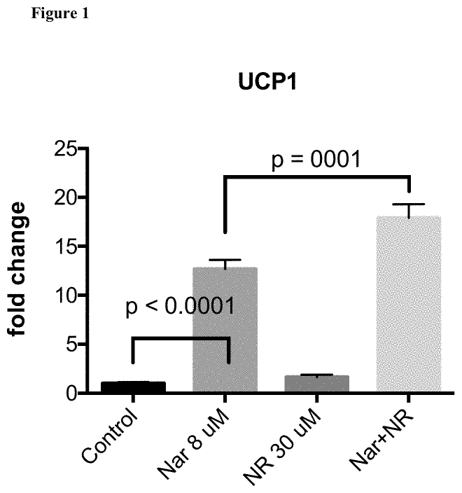

FIG. 1 shows gene expression of UCP1 in hASC cells treated with indicated treatments for 6 days. qRT-PCR from each donor cells with 10 to 12 replicates/group were performed individually. Results were combined and calculated as fold changes of Control and represented as mean±SEM. UCP1 expression was upregulated ˜12-fold by naringenin (p<0.0.0001) and this effect was increased by ˜50% with the addition of NR, resulting in an 18-fold increase in UCP1 expression (p<0.0001 vs. naringenin alone). NR exerted no independent effect on UCP 1 expression.

### Example 6: Gene Expression Results for GLUT4 with Naringenin-NR Treatment

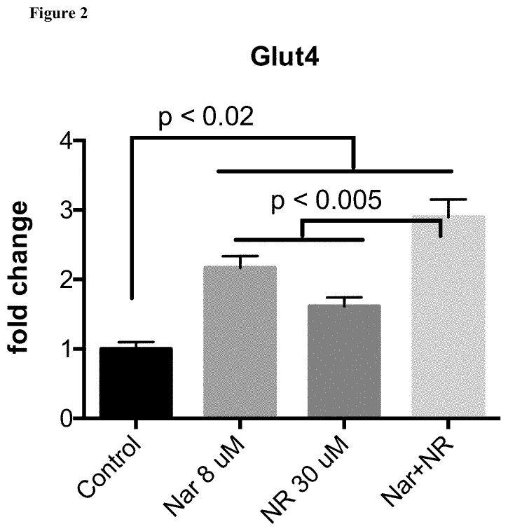

FIG. 2 shows gene expression of Glut4 in hASC cells treated with indicated treatments for 6 days. qRT-PCR from each donor cells with 10 to 12 replicates/group were performed individually. Results were combined and calculated as fold changes of Control and represented as mean±SEM. Naringenin stimulated a ˜two-fold increase in Glut4 expression (p<0.02) and the addition of NR to naringenin increased this effect to three-fold (p<0.002 vs. naringenin alone), although NR exerted no independent effect in the absence of naringenin.

### Example 7: Gene Expression Results for CPT1b with Naringenin-NR Treatment

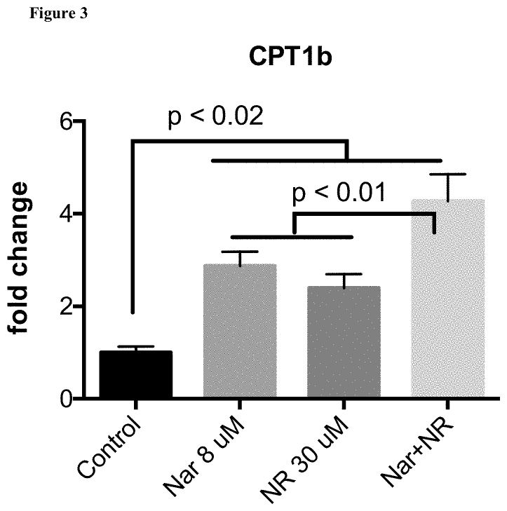

FIG. 3 shows gene expression of CPT1b in hASC cells treated with indicated treatments for 6 days. qRT-PCR from each donor cells with 10 to 12 replicates/group were performed individually. Results were combined and calculated as fold changes of Control and represented as mean±SEM. CPT1 expression was significantly increased by both naringenin and NR (˜2.4-fold, p<0.02), while the combination exerted a significantly more robust effect (4.3-fold, p<0.01 vs. naringenin or NR).

### Example 8: Gene Expression Results for ADIPOQ with Naringenin-NR Treatment

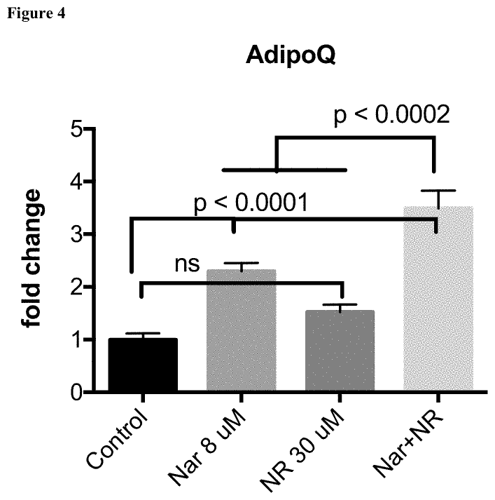

FIG. 4 shows gene expression of AdipoQ in hASC cells treated with indicated treatments for 6 days. qRT-PCR from each donor cells with 10 to 12 replicates/group were performed individually. Results were combined and calculated as fold changes of Control and represented as mean±SEM. AdipoQ expression was modestly but significantly increased by naringenin, while NR exerted no significant effect. The combination of naringenin and NR resulted in a significant 3.5-fold increase in AdipoQ vs all other treatments.

### Example 9: Gene Expression Results for ChREBP with Naringenin-NR Treatment

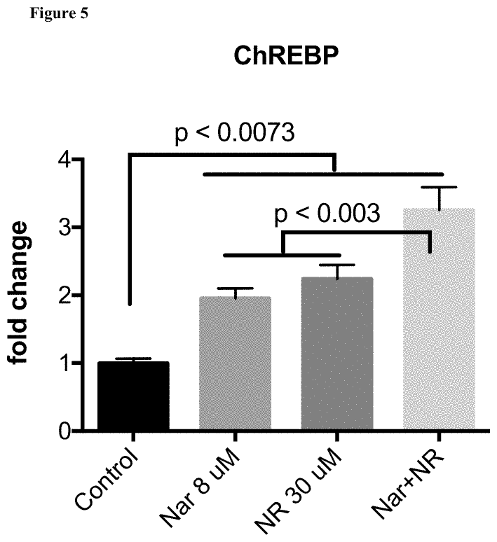

FIG. 5 shows gene expression of ChREBP in hASC cells treated with indicated treatments for 6 days. qRT-PCR from each donor cells with 10 to 12 replicates/group were performed individually. Results were combined and calculated as fold changes of Control and represented as mean±SEM. Carbohydrate-responsive element-binding protein (ChREBP) was increased ˜two-fold by naringenin and by NR independently, while the combination exerted a significantly greater 3.25-fold effect.

### Example 10: Gene Expression Results for ATGL with Naringenin-NR Treatment

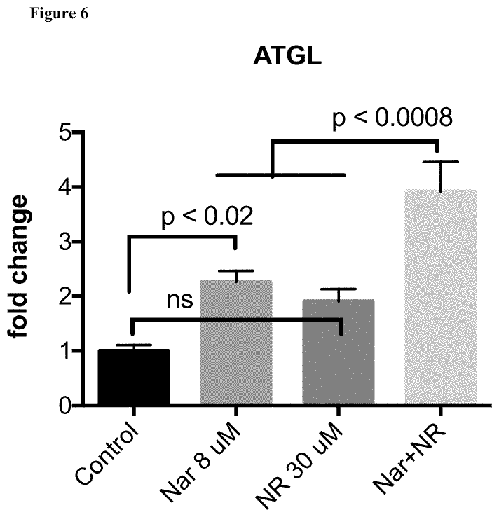

FIG. 6 shows gene expression of ATGL in hASC cells treated with indicated treatments for 6 days. qRT-PCR from each donor cells with 10 to 12 replicates/group were performed individually. Results were combined and calculated as fold changes of Control and represented as mean±SEM. Adipose triglyceride lipase (ATGL) was similarly unregulated ˜two-fold by naringenin, but not by NR. The combination of naringenin and NR resulted in a significantly greater four-fold increase in ATGL versus control.

### Example 11: Gene Expression Results for PRDM16 with Naringenin-NR Treatment

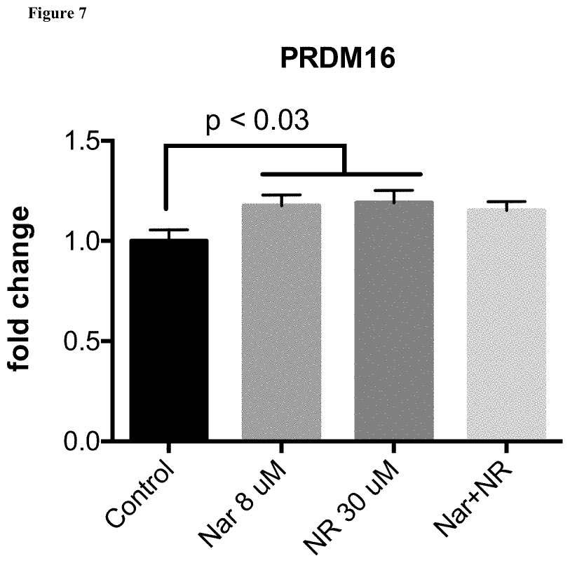

FIG. 7 shows gene expression of PRDM16 in hASC cells treated with indicated treatments for 6 days. qRT-PCR from each donor cells with 10 to 12 replicates/group were performed individually. Results were combined and calculated as fold changes of Control and represented as mean±SEM. PRDM16 expression, was significantly upregulated by Nar and NR, but there was no further augmentation of this effect by combining the two.

### Example 12: Gene Expression Results for p-AMPK and SIRT1 with Naringenin-NR Treatment

Gene expression of p-AMPK and Sirt1 was measured in hASC cells treated with indicated treatments for 6 days. p-AMPK and Sirt1 protein expression were significantly increased by Naringenin, while NR and the combination exerted no significant effect. p-AMPK was significantly increased by naringenin, but NR had no effect and there was no synergy between the two compounds.

### Example 13: Fatty Acid Oxidation Studies

Fatty acid oxidation (Seahorse) studies were conducted via the method of Example 4. Data indicate that naringenin induces an increase in both basal and palmitate-induced oxygen consumption of ˜30-40%, while a combination of naringenin with NR increases this measure of fatty acid oxidation by ˜75-90%.

### Example 14: Dosing Studies for Flavonoid/Sirtuin Pathway Activator Compositions

A dosing study of various combinations of flavonoid compounds (e.g., naringenin) and a sirtuin pathway activator (e.g., nicotinamide riboside) is conducted to determine optimal dosing combinations for affecting physiological effects such as fatty acid oxidation, fat loss or weight loss, adipocyte beiging, thermogenesis, or insulin sensitivity. The study provides data on the optimal dosage of flavonoid for producing enhanced physiological effects at a sub-therapeutic amount of sirtuin pathway activator.

Flavonoid/sirtuin pathway activator compositions are administered to human subjects. Each subject is provided a set dosage for the duration of the study. Each subject is randomly assigned a composition chosen from a) 100 mg naringenin/250 mg NR, b) 500 mg naringenin/250 mg NR, c) 1000 mg naringenin/250 mg NR, d) 100 mg naringenin/500 mg NR, e) 500 mg naringenin/500 mg NR, f) 1000 mg naringenin/500 mg NR, g) 100 mg naringenin/1000 mg NR, h) 500 mg naringenin/1000 mg NR, i) 1000 mg naringenin/1000 mg NR, and j) placebo. Compositions are administered orally twice a day for 60 days. Physiological effects, such as body weight loss, fat mass, lean mass, fat oxidation (via respiratory calorimetry) basal body temperature, and insulin level and/or blood sugar (via blood analysis) are measured in subjects on a weekly basis with the first measurement made on day 0 of the study.

### Expected Results

Subjects from group a), d) and j) are expected to show similar results for the measured physiological effects, without statistically-significant difference, after 60 days. Groups b), c), e), and f) are expected to show statistically significant differences for weight loss, fat mass loss, fat oxidation, and insulin sensitivity compared to their day 0 values. Groups g), h), and i) are expected to show statistically significant differences for weight loss, fat weight or volume loss, basal body temperature or insulin sensitivity, but a smaller difference than those observed in groups b), c), e) and f). Evidence for increased whole body fatty acid oxidation includes an increase in oxygen consumption and/or decrease in respiratory quotient (RQ), as measured by respiratory calorimetry, for subjects receiving the efficacious dosages. Evidence for increased insulin sensitivity includes a decrease in fasting insulin, a decrease in homeostatic assessment of insulin resistance (HOMA_{IR}), a decrease in 60-minute glucose or insulin in a glucose tolerance test (GTT), or a decrease in the glucose or insulin area under the curve in a GTT, as measured by blood analysis, for subjects receiving the efficacious dosages.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1. A composition comprising:
a. an amount of a flavonoid or a derivative thereof; and
b. an amount of a sirtuin pathway activator;
wherein the amount of (a) and the amount of (b) in combination are effective in enhancing in a subject at least one physiological effect selected from the group consisting of mitochondrial biogenesis, adipocyte beiging, thermogenesis, fatty acid oxidation, weight loss, fat loss, and insulin sensitivity.

2. The composition of claim 1, wherein the enhanced adipocyte beiging is evidenced by at least two-fold increase in an expression or an activity of a gene implicated in adipocyte beiging.

3. The composition of claim 2, wherein the gene implicated in adipocyte beiging is selected from the group consisting of UCP1, PRDM16, PCG1α, GLUT4, Cidea, Elovl3, Ppary, Cox8b, Dio2, Ndufs1, and Tbx1.

4. (canceled)

5. The composition of claim 1, wherein the thermogenesis is evidenced by mat least two-fold increase in an expression or an activity of a gene implicated in thermogenesis.

6. The composition of claim 5, wherein the gene implicated in thermogenesis is selected from the group consisting of UCP1, PCG1α, PRDM16, PCG1β, Ppary, COX-2, and Cidea.

7. (canceled)

8. The composition of claim 1, wherein the enhanced fatty acid oxidation is evidenced by at least two-fold increase in an expression or an activity of a gene implicated in fatty acid oxidation, weight loss, or fat loss.

9. The composition of claim 8, wherein the gene implicated in fatty acid oxidation, weight loss, or fat loss is selected from the group consisting of CPT1β, AdipoQ, ChREBP, ATGL, UCP2, CPT1α, and NPY.

10. The composition of claim 1, wherein the enhanced fatty acid oxidation is evidenced by an increase in oxygen consumption or a decrease in respiratory quotient, as measured by respiratory calorimetry.

11. (canceled)

12. The composition of claim 1, wherein the enhanced insulin sensitivity is evidenced by at least two-fold increase in an expression or an activity of a gene implicated in insulin sensitivity.

13. The composition of claim 12, wherein the gene implicated in insulin sensitivity is selected from the group consisting of GLUT4, AdipoQ, and ChREBP.

14. The composition of claim 1, wherein the enhanced insulin sensitivity is evidenced by a decrease in fasting insulin, a decrease in homeostatic assessment of insulin resistance (HOMA_{IR}), a decrease in 60-minute glucose or insulin in a glucose tolerance test (GTT), or a decrease in the glucose or insulin area under the curve in a GTT.

15. (canceled)

16. The composition of claim 1, wherein the administration of (a) and (b) is in an amount that synergistically enhances at least one of the effects selected from adipocyte beiging, thermogenesis, insulin sensitivity, fatty acid oxidation, weight loss, or fat loss.

17. (canceled)

18. (canceled)

19. (canceled)

20. The composition of claim 1, wherein the flavonoid or derivative thereof comprises a flavanone compound comprising a compound selected from the group consisting of blumeatin, butin, eriodictyol, hesperetin, hesperidin, homoeriodictyol, isosakuranetin, naringenin, naringin, pinocembrin, poncirin, sakuranetin, sakuranin, sterubin, and/or pinostrobin.

21. (canceled)

22. The composition of claim 1, wherein the sirtuin pathway activator comprises nicotinamide riboside, nicotinamide mononucleotide, nicotinic acid, or a nicotinic acid metabolite.

23. The composition of claim 22, wherein the nicotinic acid metabolite comprises nicotinyl CoA, nicotinuric acid, nicotinate mononucleotide, nicotinate adenine dinucleotide, or nicotinamide adenine dinucleotide.

24. The composition of claim 1, wherein the flavonoid comprises naringenin and the sirtuin pathway activator comprises nicotinamide riboside.

25. The composition of claim 20, wherein the molar ratio of (a) to (b) is at least 3:1.

26. (canceled)

27. The composition of claim 1, wherein the amount of (b) is a sub-therapeutic amount when administered alone to the subject.

28. The composition of claim 1, wherein the sirtuin pathway activator is substantially free of nicotinamide.

29.-82. (canceled)

83. The composition of claim 1, wherein the amount of a flavonoid or a derivative thereof ranges from 10 mg to 2000 mg, and/or the amount of a sirtuin pathway activator ranges from 100 mg to 2000 mg.
